(19) United States
(12) Patent Application Publication
(10) Pub. No.: US 2015/0209198 A1
(43) Pub. Date: Jul. 30, 2015

(54) Selective Wetting and Transport Surfaces

(71) Applicant: President and Fellows of Harvard College, Cambridge, MA (US)

(72) Inventors: Joanna Aizenberg, Boston, MA (US); Tak Sing Wong, State College, PA (US); Benjamin Hatton, Toronto (CA)

(21) Appl. No.: 14/414,592

(22) PCT Filed: Jul. 12, 2013
(86) PCT No.: PCT/US2013/050402

§ 371 Date: Jan. 13, 2015

Related U.S. Application Data
(60) Provisional application No. 61/671,442, filed on Jul. 13, 2012.
(60) Provisional application No. 61/671,645, filed on Jul. 13, 2012.
(60) Provisional application No. 61/673,705, filed on Jul. 19, 2012.
(60) Provisional application No. 61/746,296, filed on Dec. 27, 2012.

(51) Int. Cl. A61F 13/537; C10M 107/38; C09D 171/00; A61F 13/15

(52) U.S. Cl. CPC A61F 13/537; A61F 13/15699; A61F 13/15617; C10M 107/38; C09D 171/00; A61F 2013/8455

(57) ABSTRACT

Methods and compositions disclosed herein relate to liquid repellant surfaces having selective wetting and transport properties.

### CROSS-REFERENCE TO RELATED APPLICATIONS

The present application claims priority to U.S. Patent Application No. 61/671,442, filed Jul. 13, 2012; U.S. Patent Application No. 61/671,645, filed Jul. 13, 2012; U.S. Patent Application No. 61/673,705, filed Jul. 19, 2012; and U.S. Patent Application No. 61/746,296, filed Dec. 27, 2012, the entire contents of which are hereby incorporated by reference.

This application is related to the following co-pending applications filed on even date herewith:

International Patent Application entitled SLIPS SURFACE BASED ON METAL-CONTAINING COMPOUND;

International Patent Application entitled MULTIFUNCTIONAL REPELLENT MATERIALS;

International Patent Application entitled STRUCTURED FLEXIBLE SUPPORTS AND FILMS FOR LIQUID-INFUSED OMNIPHOBIC SURFACES; and

International Patent Application entitled SLIPPERY LIQUID-INFUSED POROUS SURFACES HAVING IMPROVED STABILITY, which are incorporated in their entirety by reference.

### STATEMENT OF GOVERNMENT SUPPORT

This invention was made with government support under N66001-11-1-4180 awarded by the U.S. Department of Defense. The government has certain rights in the invention.

### TECHNICAL FIELD

The field of this application generally relates to slippery surfaces, methods for forming them, and their uses.

### BACKGROUND

Current development of liquid-repellent surfaces is inspired by the self-cleaning abilities of many natural surfaces on animals, insects, and plants. Water droplets on these natural surfaces roll off or slide off easily, carrying the dirt or insects away with them. The presence of the micro/nanostructures on many of these natural surfaces has been attributed to the water-repellency function. These observations have led to enormous interests in manufacturing biomimetic water-repellent surfaces in the past decade, owing to their broad spectrum of potential applications, ranging from water-repellent fabrics to friction-reduction surfaces. Previously it has been challenging to provide a non-wetting, water-repellant surface that can also allow selective wetting and transport of fluids, which is important for certain applications such as lab-on-a-chip devices or wound dressings that require a non-stick, hydrophobic surface and an ability to drain exudate fluids. Bandages and wound dressings often have a problem of adhesion to the wound tissue, causing damage upon removal. There is a need for a non-adherent wound dressing that can allow absorbent wicking, and oxygen permeability.

### SUMMARY

Liquid-repellant surfaces having selective wetting and transport properties are described.

In one aspect a liquid-coated substrate includes a substrate having a spatially heterogeneous surface pattern. The spatially heterogeneous pattern includes at least one first region having surface characteristics that provide a stable liquid film with a first lubricating liquid; and at least one second region having one of surface characteristics that provide one of an unstable liquid film with said first lubricating liquid or apertures or holes that provide liquid conduits through the substrate; and a first lubricating liquid disposed over at least a portion of said first region to provide an immobilized liquid layer.

In one or more embodiments, the liquid-coated substrate is a non-adherent article capable of absorbent wicking, wherein the spatially heterogeneous surface pattern is disposed through at least a portion of the thickness of the substrate; wherein the first lubricating liquid is substantially immobilized in and on the at least one first region of the substrate to form a stabilized liquid overlayer, said overlayer providing a non-adherent surface; wherein the first lubricating liquid has a lower affinity for the second region of the substrate as compared to the at least one first region or the second region comprises a hole or aperture that does not retain the first lubricating liquid; and wherein the second region is capable of wicking a wicking liquid into or through said second region.

In any preceding embodiment, the second region includes apertures or holes.

In one or more embodiments, the substrate comprises a perforated material with large holes that provide liquid conduits through the material.

In one or more embodiments, the substrate is selected from a flat solid, a textured solid, a porous solid and combinations thereof.

In one or more embodiments, at least one of the first region or second region is chemically functionalized to provide surface characteristics that provide either a stable liquid film or an unstable liquid film with the first lubricating liquid.

In one or more embodiments, the article further includes an absorbent backing.

In one or more embodiments, the wicking liquid is immiscible in the first lubricating liquid.

In one or more embodiments, the wicking liquid has a greater affinity for the apertures than the first lubricating liquid.

In one or more embodiments, wicking liquid comprises at least one of proteins, bacteria, cells or non-biological entities.

In one or more embodiments, the first region is hydrophobic, and the second region comprises chemically etched hydrophilic regions or holes allows the wicking and transport of an aqueous phase.

In one or more embodiments, the substrate is porous and wherein the second region is capable of wicking a wicking liquid into or through said porous substrate.

In one or more embodiments, the article is configured and arranged to collect the absorbed/wicked liquid without the removal of the article.

In one or more embodiments, the article is configured and arranged to inject/wick through the conduits certain liquids to reach the target area without the removal of the article.

In any preceding embodiment, the apertures have an average diameter or width of at least about 200 nm, or at least about 0.2 mm, or at least about 0.25 mm, at least about 0.5 mm, at least about 0.75 mm, at least about 1.00 mm, at least about 1.25 mm, at least about 1.50 mm, at least about 1.75 mm, or at least about 2.00 mm.

In any preceding embodiment, the liquid-coated substrate is incorporated into a wound dressing, diaper, surgical dressing, sanitary pad or antiperspirant dressing.

In any preceding embodiment, the article comprises a liquid-coated substrate surrounded by an adhesive area.

In any preceding embodiment, spatially heterogeneous surface is non-adhesive to at least one of skin, hair, dried blood or clotted blood.

In any preceding embodiment, the liquid-coated substrate is anti-bacterial, or the liquid-coated substrate is pathogen-resistant, or the liquid-coated substrate is anti-adhesion against biological cells, or the liquid-coated substrate has anti-coagulating properties.

In one or more embodiments, the substrate is a flat solid, the at least one first region comprising chemical functionalization.

In one or more embodiments, the substrate is a roughened solid, the at least one first region comprising at least one of chemical functionalization or the roughened solid.

In one or more embodiments, the substrate is a porous solid, the at least one first region comprising at least one of chemical functionalization or the porous solid.

In one aspect, a liquid-coated substrate, wherein the liquid-coated substrate is a non-adhesive article capable of absorbent wicking. The liquid-coated substrate includes a substrate; at least one first region disposed on an upper portion of the substrate, said at least one first region having surface characteristics that provide a stable liquid film with a first lubricating liquid; at least one second region disposed on the lower portion of the substrate, said at least one second region having surface characteristics that provide an unstable liquid film with said first lubricating liquid, wherein the at least one second region is capable of wicking a wicking liquid into or through said second region; a first lubricating liquid disposed over at least a portion of said at least one first region, wherein:
the first lubricating liquid is substantially immobilized in and on the at least one first region of the substrate to form a stabilized liquid overlayer, said overlayer providing a non-adherent surface;
the first lubricating liquid has a lower affinity for the at least one second region as compared to the at least one first region; and
said wicking liquid is immiscible in the first lubricating liquid and has a lower affinity for the at least one first region as compared to the at least one second region.

In one or more embodiments, the upper porous layer of the substrate is hydrophobic, oleophobic or omniphobic and the lower porous layer of the substrate is hydrophilic or oleophilic or omniphilic.

In one or more embodiments, the second liquid comprises a curable polymer.

In one or more embodiments, the second liquid comprises an adhesive.

In one or more embodiments, the liquid-coated substrate further includes a composite backing layer comprising a solid polymer infused in the lower porous layer of the substrate.

In one or more embodiments, the first lubricating liquid is optical refractive index-matched with the substrate.

In one or more embodiments, at least one of the first lubricating liquid or the second liquid is transparent to at least one of infrared, visible, or ultra-violet lights and wherein at least one of the first lubricating liquid or the second liquid is opaque to at least one of infrared, visible, or ultra-violet lights.

In one or more embodiments, the liquid-coated substrate is suitable as an optical filter.

In one or more embodiments, the spatially heterogeneous surface pattern comprises at least one of a predetermined pattern, a symbol or a drawing.

In one or more embodiments, the upper layer includes a spatially heterogeneous surface pattern, said spatially heterogeneous pattern comprising at least one first region having surface characteristics that provide a stable liquid film with the first lubricating liquid.

In one or more embodiments, the spatially heterogeneous pattern of the upper layer further includes at least one second region having surface characteristics that provide an unstable liquid film with the first lubricating liquid.

In one or more embodiments, at least a portion of said lower layer having affinity for an adhesive.

In one or more embodiments, at least one of the first side or the second side comprises an adhesive.

In one aspect, a liquid-coated substrate is configured as a bandage to be applied to a wound, wherein the liquid-coated substrate is a non-adhesive article capable of absorbent wicking. The liquid-coated substrate includes a substrate having a spatially heterogeneous surface pattern disposed through at least a portion of the thickness of the substrate, The spatially heterogeneous pattern includes at least one first region having surface characteristics that provide a stable liquid film with a first lubricating liquid; at least one second region having surface characteristics that provide an unstable liquid film with said first lubricating liquid; and a first lubricating liquid disposed over at least a portion of said first region to provide an immobilized liquid layer; an absorbent backing; and a protective overlayer to protect the wound from the environment and to adhere to healthy tissue surrounding the wound; wherein the first lubricating liquid is substantially immobilized in and on the at least one first region of the substrate to form a stabilized liquid overlayer, said overlayer providing a non-adherent surface; wherein the first lubricating liquid has a lower affinity for the second region of the porous substrate as compared to the at least one first region; and wherein the second region is capable of wicking a wicking liquid into or through said second region.

In one aspect, a liquid-coated substrate is configured as fluid-transport devices, wherein the liquid-coated substrate is a non-adhesive article capable of fluid transport for biomedical and diagnostic purposes. The liquid-coated substrate includes:

a substrate having a spatially heterogeneous surface pattern disposed through at least a portion of the thickness of the substrate, said spatially heterogeneous pattern comprising:
at least one first region having surface characteristics that provide a stable liquid film with a first lubricating liquid;
at least one second region having surface characteristics that provide an unstable liquid film with said first lubricating liquid; and
a first lubricating liquid disposed over at least a portion of said first region to provide an immobilized liquid layer;

wherein the first lubricating liquid is substantially immobilized in and on the at least one first region of the substrate to form a stabilized liquid overlayer, said overlayer providing a non-adherent surface;

wherein the first lubricating liquid has a lower affinity for the second region of the porous/roughened substrate as compared to the at least one first region; and
wherein the second region is capable of transporting fluids into or through said second region.

In one aspect, a liquid-coated substrate is configured as selective patterning devices for biological species, such as proteins, DNA, RNA, viruses, cells, tissues, etc., wherein the liquid-coated substrate is a non-adhesive article capable of preventing adhesion from biological species. The liquid-coated substrate includes:

a substrate having a spatially heterogeneous surface pattern disposed through at least a portion of the thickness of the substrate, said spatially heterogeneous pattern comprising:
at least one first region having surface characteristics that provide a stable liquid film with a first lubricating liquid;
at least one second region having surface characteristics that provide an unstable liquid film with said first lubricating liquid; and
a first lubricating liquid disposed over at least a portion of said first region to provide an immobilized liquid layer;

wherein the first lubricating liquid is substantially immobilized in and on the at least one first region of the substrate to form a stabilized liquid overlayer, said overlayer providing a non-adherent surface;

wherein the first lubricating liquid has a lower affinity for the second region of the porous/roughened substrate as compared to the at least one first region; and
wherein the second region is capable of selectively patterning biological species into or through said second region.
   In another aspect, a method for absorbing a fluid, includes:

providing the non-adhesive liquid-coated substrate of any of claims 1-38; and

exposing the liquid-coated substrate to a third liquid to be absorbed that is immiscible with the first lubricating liquid, wherein the third liquid fills the one or more apertures or second region of the liquid-coated substrate without displacing the stabilized liquid overlayer of the liquid-coated substrate to thereby retain the low adhesion properties of the liquid-coated substrate during absorption.

In one or more embodiments, the liquid-coated substrate is exposed to a wound.

In one or more embodiments, the method further includes disposing an oxygen-permeable lubricant between said liquid-coated substrate and said wound.

In one or more embodiments, the lubricant is selected from perfluorocarbons and silicone oils.

In another aspect, a method of adhering a liquid-coated substrate to an exposed surface of an object includes:
(a) providing a liquid-coated substrate, the liquid-coated substrate comprising a first region; the first region having surface characteristics that provide a stable liquid film with a first lubricating liquid; a first lubricating liquid disposed over at least a portion of said first region; and a second region surround the first region, the second region having surface characteristics that provide an adhesive; and
(b) contacting the portion of the liquid-coated substrate containing the first region to an exposed surface of the object, said contacting causing the second region of the liquid-coated substrate to adhere to the exposed surface of the object.

In some embodiments, the first region is hydrophobic and the second region is hydrophilic. In further embodiments, the first region is lipophobic and the second region is lipophilic. In still further embodiments, the first region is oleophobic and the second region is oleophilic. In still further embodiments, the first region or the second region is omniphobic. In still further embodiments, the first region or the second region is omniphilic. In still further embodiments, the first region is adhesive to a target surface and the second region is non-sticky.

In one aspect, a method of making a liquid-coated substrate is disclosed, the liquid-coated substrate comprising: a substrate having a spatially heterogeneous surface pattern, said spatially heterogeneous pattern comprising: at least one first region having surface characteristics that provide a stable liquid film with a first lubricating liquid; and at least one second region having surface characteristics that provide an unstable liquid film with said first lubricating liquid; and a first lubricating liquid disposed over at least a portion of said first region to provide an immobilized liquid layer; the method comprising chemically functionalizing the substrate to provide a first region characterized at least in part by surface characteristics that provide a stable liquid film with the first lubricating liquid.

In some embodiments, the substrate is porous/roughened and the second region of the substrate is formed by physical or chemical etching to provide a second region having at least in part surface characteristics that provide an unstable film with the first lubricating liquid.

In further embodiments, the second region of the substrate is formed by chemically functionalizing the substrate and subsequently removing the chemical functionalization in selected regions to provide a chemically functionalized first region having at least in part surface characteristics that provide a stable film with the first lubricating liquid and a second region that is substantially free of chemical functionalization.

In some embodiments, the chemical functionalization is removed by at least one of physical etching, chemical etching, oxygen plasma or use of a mask. In some embodiments, the liquid-coated substrate is formed by masking the substrate to provide exposed regions and masked regions, and chemically functionalizing the exposed regions of the substrate to form the first region of the substrate, said chemical functionalization providing a first region having at least in part surface characteristics that provide a stable film with the first lubricating liquid, and a second region that is free of chemical functionalization. In some embodiments, chemical functionalization is accomplished by vapor or liquid phase processes. In some embodiments, the substrate is chemically functionalized with moieties that provide high affinity to lubricating liquid, such as functionalized with heptadecafluoro-1,1,2,2-tetrahydrodecyltrichlorosilane when hydrophobic and fluorinated lubricants are used.

In some embodiments, the first region of the substrate is formed by physically roughening the substrate to provide at least in part surface characteristics that provide a stable liquid film with the first lubricating liquid. In some embodiments, the physically roughening comprises at least one of additive or subtractive processes. In further embodiments, the process is subtractive and is selected from at least one of sand-blasting, sputtering, reactive ion etching, or chemical etching. In still further embodiments, the process is chemical transformation. In still further embodiments, the chemical transformation comprises boehmite formation from an aluminum oxide substrate. In still further embodiments, the process is additive and is selected from at least one of sol-gel deposition, colloidal deposition, growth of inorganic nanostructures (such as carbon nanotubes), growth of polymeric nanostructures (such as polypyrrole), particles spraying, layer-by layer deposition, or the formation of nanostructures. In still further embodiments, the substrate is patterned by shadow masking, photolithography or soft lithography. In still further embodiments, the liquid-coated substrate is formed by masking the substrate to provide exposed regions and masked regions, and chemically functionalizing the exposed regions of the substrate to form the first region of the substrate, said chemical functionalization providing a first region having at least in part surface characteristics that provide an unstable film with the first lubricating liquid and a second region that is free of chemical functionalization, yet the intrinsic material has strong affinity with the first lubricating liquid to form a stable film. In still further embodiments, the liquid-coated substrate comprises at least one of holes, apertures, pores, channels, wells, voids or perforations, the method further comprising punching apertures in the substrate.

### BRIEF DESCRIPTION OF THE DRAWINGS

The following figures are provided for the purpose of illustration only and are not intended to be limiting.

FIG. 1 shows a schematic of a self-healing slippery liquid-infused porous surface (SLIPS) in accordance with certain embodiments of the present disclosure.

FIG. 2 shows an illustration of a method of creating SLIPS and non-SLIPS regions by controlling the interfacial energies of the solid surface with a suitable lubricant to create stable and unstable regions with region-specific properties in accordance with certain embodiments of the present disclosure.

FIG. 3 shows a schematic for formation of patterned absorbing material in accordance with certain embodiments of the present disclosure.

FIG. 4 shows a schematic for formation of a 2-layer porous solid that is composed of two different types of materials in accordance with certain embodiments of the present disclosure.

FIG. 5 shows a schematic for the preparation of a patterned SLIPS on a flat, smooth solid in accordance with certain embodiments of the present disclosure.

FIG. 6 shows a schematic for the preparation of a patterned SLIPS on a 2.5 D patterned solid (i.e. beginning with a smooth substrate) in accordance with certain embodiments of the present disclosure.

FIG. 7 shows a schematic for the preparation of a patterned SLIPS on a 3D porous solid (i.e. beginning with a porous substrate) in accordance with certain embodiments of the present disclosure.

FIG. 8 shows an alternative schematic for the preparation of a patterned SLIPS on a 3D porous solid in accordance with certain embodiments of the present disclosure.

FIG. 9 shows an alternative schematic for the preparation of a patterned SLIPS on a 3D porous solid in accordance with certain embodiments of the present disclosure

FIGS. 10A through 10C shows a diagram of a patterned SLIPS layer allowing an aqueous phase to pass through a perforated SLIPS layer when placed into contact with the surface in accordance with certain embodiments of the present disclosure.

FIG. 11 shows a schematic image of a SLIPS wound dressing or bandage in accordance with certain embodiments of the present disclosure.

FIG. 12 contains an SEM image showing the photo-patterned structured boehmite (AlO(OH)) coating on a glass substrate produced from UV-sensitive sol-gel alumina precursors and the resulting boehmite morphology.

FIG. 13 shows an example of a second liquid displacing a first liquid in a heterogeneously functionalized SLIPS substrate in accordance with certain embodiments of the present disclosure.

FIG. 14 shows SEM images of inverse monolayer structures prepared from colloids with sizes as specified in the insets in accordance with certain embodiments of the present disclosure.

FIG. 15 shows a schematic illustration for forming a patterned SLIPS surface over surface features USING inverse monolayer structures in accordance with certain embodiments.

FIG. 16 shows patterned transparent SLIPS coatings generated by selective colloidal deposition in accordance with certain embodiments of the present disclosure.

FIG. 17A-17D shows a series of images demonstrating a SLIPS membrane (ePTFE saturated with perfluorocarbon liquid) which contains an array of 1 mm diameter holes coupled with an absorbent backing layer (hydrophilic tissue) used as a bandage in which FIG. 17A shows an aqueous test fluid, FIG. 17B shows the SLIPS membrane in contact with the test fluid, whereby the bandage immediately causes wicking through the SLIPS layer, to wet only the absorbing layer, FIG. 17C shows the separated absorbing material, which contains the test fluid, while FIG. 17D shows that the SLIPS layer has not absorbed any of the test fluid.

### DETAILED DESCRIPTION

The patent and scientific literature referred to herein establishes knowledge that is available to those of skill in the art. The issued U.S. patents, allowed applications, published foreign applications, and references, that are cited herein are hereby incorporated by reference to the same extent as if each was specifically and individually indicated to be incorporated by reference.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The present disclosure describes slippery surfaces referred to herein as Slippery Liquid-Infused Porous Surfaces (SLIPS). In certain embodiments, the slippery surfaces of the present disclosure exhibit anti-adhesive and anti-fouling properties. The slippery surfaces of the present disclosure are able to prevent adhesion of a wide range of materials. Exemplary materials that do not stick onto the surface include liquids, solids, and gases (or vapors). For example, liquids such as water, oil-based paints, hydrocarbons and their mixtures, organic solvents, complex fluids such as crude oil, protein-containing fluids and the like can be repelled. As another example, complex biological fluids, such as bodily fluids (blood, saliva, urine, secretions, mucus, and the like) can be repelled. The liquids can be both pure liquids and complex fluids. In certain embodiments, SLIPS can be designed to be omniphobic, where SLIPS exhibit both hydrophobic and oleophobic properties. As another example, solids such as bacteria, spores, insects, fungi, algae, and the like can be repelled. As another example, solids such as ice, paper, sticky notes, or inorganic particle-containing paints, dust particles can be repelled or cleaned. SLIPS surfaces are discussed in International Patent Application Nos. PCT/US2012/21928 and PCT/US2012/21929, both filed Jan. 19, 2012, the contents of which are hereby incorporated by reference in their entireties.

Such materials that can be prevented from sticking to the slippery surfaces disclosed herein are referred to herein as “Object A.” Object A that is in liquid form is referred to as “Object A in liquid form,” or “liquefied Object A,” or “Liquid A.” Object A that is in solid form is referred to as “Object A in solidified form,” or “solidified Object A” or “Solid A.” In certain embodiments, Object A can contain a mixture of both solids and fluids.

A wide range of materials can be repelled by the slippery surfaces of the present disclosure. For example, Object A can include polar and non-polar Liquids A and their solidified forms, such as hydrocarbons and their mixtures (e.g., from pentane up to hexadecane and mineral oil, paraffinic extra light crude oil; paraffinic light crude oil; paraffinic light-medium crude oil; paraffinic-naphthenic medium crude oil; naphthenic medium-heavy crude oil; aromatic-intermediate medium-heavy crude oil; aromatic-naphthenic heavy crude oil, aromatic-asphaltic crude oil, etc.), ketones (e.g., acetone, etc.), alcohols (e.g., methanol, ethanol, isopropanol, dipropylene glycol, ethylene glycol, and glycerol, etc.), water (with a broad range of salinity, e.g., sodium chloride from 0 to 6.1 M; potassium chloride from 0 to 4.6 M, etc.), acids (e.g., concentrated hydrofluoric acid, hydrochloric acid, nitric acid, etc.) and bases (e.g., potassium hydroxide, sodium hydroxide, etc.), and ice, etc. Object A can include biological objects, such as insects, small animals, protozoa, bacteria, viruses, fungi, bodily fluids and tissues, proteins and the like. Object A can include solid particles suspended in liquid. Object A can include non-biological objects, such as dust, colloidal suspensions, spray paints, food items, common household materials, and the like. Object A can include adhesives and adhesive films. The list is intended to be exemplary and the slippery surfaces of the present disclosure are envisioned to successfully repel numerous other types of materials.

In certain embodiments, the slippery surface of the present disclosure has a coefficient of friction that is lower than that of polytetrafluoroethylene (PTFE or TEFLON) surface. In certain embodiments, the coefficient of friction may be less than 0.1, less than 0.05, or even less than 0.04. In certain embodiments, the coefficient of friction can be measured by sliding two different surfaces against each other. The value of the coefficient will depend on the load applied onto the surfaces, the sliding velocity, and the materials of the surfaces. For example, a reference surface, such as a polished steel, could be used to slide against the target surfaces, such as Teflon, or the SLIPS of the present disclosure could be used to slide against itself (e.g., SLIPS/SLIPS) to obtain the coefficients of friction (both static and dynamic).

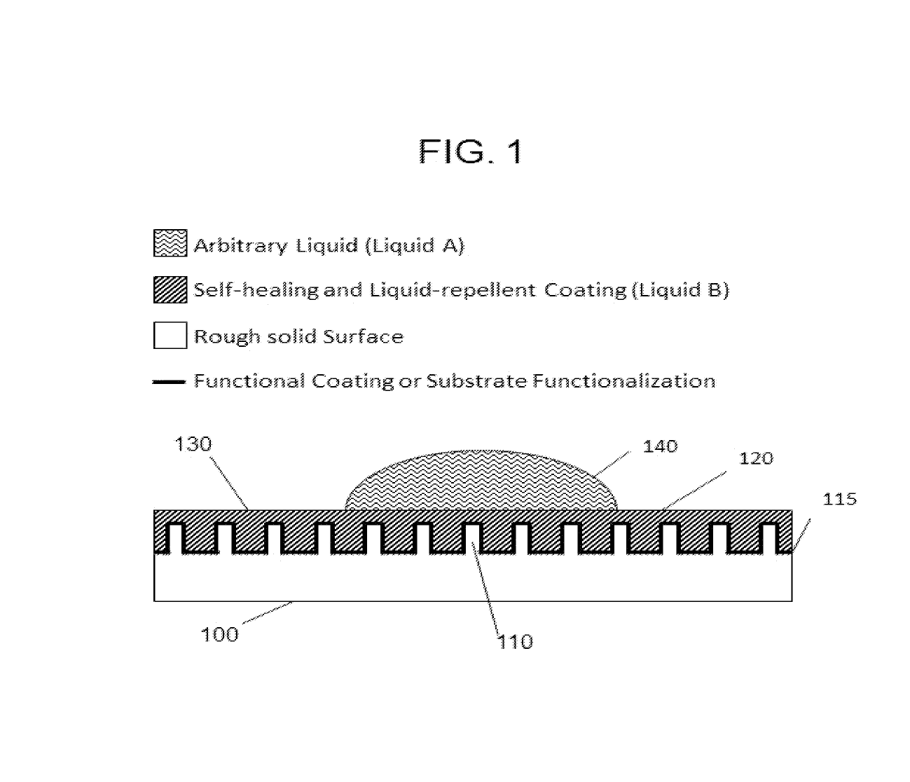

A schematic of the overall design of Slippery Liquid-Infused Porous Surfaces (SLIPS) is illustrated in FIG. 1. As shown, the article includes a solid surface 100 having surface features 110 that provide a certain roughness (i.e. roughened surface) with Liquid B 120 applied thereon. A functional layer 115 can be applied to the substrate surface 100/110 to provide affinity of Liquid B for the surface. Liquid B wets the roughened surface, filling the hills, valleys, and/or pores of the roughened surface, and forming an ultra-smooth surface 130 over the roughened surface. This surface can either create an overcoat layer covering the topography of the substrate, or form a conformal coating that follows the topography of the structured surface. Due to the ultra-smooth surface resulting from wetting the roughened surface with Liquid B. Object A 140 does not adhere to the surface.

SLIPS can be designed based on the surface energy matching between a lubricating fluid and a solid (i.e. formation of a stable lubricating film which is not readily displaced by other, immiscible fluids). In some embodiments, SLIPS can be designed based on at least the following three factors: 1) the lubricating liquid (Liquid B) can infuse into, wet, and stably adhere within the roughened surface, 2) the roughened surface can be preferentially wetted by the lubricating liquid (Liquid B) rather than by the liquid, complex fluids or undesirable solids to be repelled (Object A), and 3) the lubricating fluid (Liquid B) and the object or liquid to be repelled (Object A) can be immiscible and may not chemically interact with each other. These factors can be designed to be permanent or lasting for time periods sufficient for a desired life or service time of the SLIPS surface or for the time till a reapplication of the partially depleted infusing liquid is performed.

The first factor (a lubricating liquid (Liquid B) which can infuse into, wet, and stably adhere within the roughened surface) can be satisfied by using micro- and/or nanotextured, rough substrates whose large surface area, combined with chemical affinity for Liquid B, facilitates complete wetting by, and adhesion of, the lubricating fluid. More specifically, the roughness of the roughened surface, R, can be selected such that R≧1/cos θ_{BX}, where R is defined as the ratio between the actual and projected areas of the surface, and θ_{BX} is the equilibrium contact angle of Liquid B on a flat solid substrate immersed under medium X (X=water/air/other immiscible fluid medium). In certain embodiments, R may be any value greater than or equal to 1, such as 1 (this will correspond to a flat, unstructured surface), 1.5, 2, 5, or higher.

To satisfy the second factor (that the roughened surface can be preferentially wetted by the lubricating liquid (Liquid B) rather than by the liquid, complex fluids or undesirable solids to be repelled (Object A)), a determination of the chemical and physical properties required for working combinations of substrates and lubricants can be made. This relationship can be qualitatively described in terms of affinity; to ensure that the Object A to be repelled (fluid or solid) remains on top of a stable lubricating film of the lubricating liquid, the lubricating liquid must have a higher affinity for the substrate surface than materials to be repelled, such that the lubricating layer cannot be displaced by the liquid or solid to be repelled. This relationship can be described as a “stable” region. As stated above, these relationships for a “stable” region can be designed to be satisfied permanently or for a desired period of time, such as lifetime, service time, or for the time till the replenishment/reapplication of the partially depleted infusing liquid is performed.

A comparison of the total interfacial energies between textured solids that are completely wetted by either an arbitrary immiscible liquid (E_{A}), or a lubricating fluid with (E₁) or without (E₂) a fully wetted immiscible test liquid floating on top of it can be calculated. This can ensure that Object A remains on top of a stable lubricating film of Liquid B. In order to ensure that the solid is wetted preferentially by the lubricating fluid, both ΔE₁=E_{A}−E₁>0 and ΔE₂=E_{A}−E₂>0 should be true. The equations can be expressed as:

ΔE₁=R(γ_{B} cos θ_{B}−γ_{A} cos θ_{A})−γ_{AB}>0 (eq. 1)

ΔE₂=R(γ₂ cos θ_{B}−γ_{A} cos θ_{A})+γ_{A}−γ_{B}>0 (eq. 2)

where γ_{A} and γ_{B} are the surface tensions for the test liquid to be repelled and the lubricating fluid, γ_{AB} is the interfacial tension at the liquid-liquid interface, θ_{A} and θ_{B} are the equilibrium contact angles of the immiscible test liquid and the lubricating fluid on a flat solid surface, and R is the roughness factor (i.e. the ratio between the actual and projected surface areas of the textured solids). This relationship can also be qualitatively described in terms of affinity; to ensure that Object A remains on top of a stable lubricating film of Liquid B, Liquid B must have a higher affinity for the substrate than Object A. This relationship can be described as a “stable” region. Conversely, where Object A has a higher affinity for the substrate (for example, and unfunctionalized region of the substrate) than Liquid B, Object A will displace Liquid B in that region. This relationship can be described as an “unstable” region.

To satisfy the third factor (that the lubricating fluid (Liquid B) and the object or liquid to be repelled (Object A) can be immiscible and may not chemically interact with each other), the enthalpy of mixing between Object A and Liquid B should be sufficiently high (e.g., water/oil; insect/oil; ice/oil, etc.) that they phase separate from each other when mixed together, and/or do not undergo substantial chemical reactions between each other. In certain embodiments, Object A and Liquid B are substantially chemically inert with each other so that they physically remain distinct phases/materials without substantial mixing between the two. For excellent immiscibility between Liquid A and Liquid B, the solubility in either phase should be <500 parts per million by weight (ppmw). For example, the solubility of water (Liquid A) in perfluorinated fluid (Liquid B, e.g., 3M Fluorinert™) is on the order of 10 ppmw; the solubility of water (Liquid A) in polydimethylsiloxane (Liquid B, MW=1200) is on the order of 1 ppm. In some cases, SLIPS performance could be maintained transiently with sparingly immiscible Liquid A and Liquid B. In this case, the solubility of the liquids in either phase is <500 parts per thousand by weight (ppthw). For solubility of >500 ppthw, the liquids are said to be miscible. For certain embodiments, an advantage can be taken of sufficiently slow miscibility or mutual reactivity between the infusing liquid and the liquids or solids or objects to be repelled, leading to a satisfactory performance of the resulting SLIPS over a desired period of time.

A meta-stable state is created when the lubricant's low surface tension wets the surface but a “lock in”, that is, the energetical minimum situation is not supported by the surface chemistry. As a result, the SLIPS state will eventually break down upon addition of a second liquid that has a higher affinity to the underlying surface. However, this may take time, and the surface will remain in a SLIPS state until the stable surface lubricating layer is disrupted. Thus, a meta-stable slips surface can be created even though the conditions for thermodynamic stability are not satisfied. A meta-stable state could also be created on a surface on which the supporting roughness is not high enough to allow a stabilized liquid layer (SLIPS) to form. The formation of such meta-stable slips surface is still advantageous over non-slippery materials, if it can maintain its slippery properties for a desired period of time, such as lifetime, service time, or for the time till the replenishment/reapplication of the partially depleted infusing liquid is performed.

The properties of the SLIPS surfaces disclosed herein, namely, an ultra-smooth surface resulting from wetting the roughened surface with the wetting liquid, such that other liquids, solids and gases do not adhere to the surface, make it suitable for a bandage or wound dressing. The SLIPS surface can provide protection without sticking to the wound. However, the high slip and low adhesive properties of SLIPS surfaces provides challenges as a wound dressing. The same repellant surface that prevents adhesion to the wound, also prevents absorption of wound exudates. In addition, the low adhesive properties of SLIPS surfaces makes it difficult to adhere such surfaces to other layers, such as a backing layer. Accordingly, disclosed herein are articles exhibiting different properties on different surfaces of the article. e.g., different surfaces having slip and non-slip properties.

Further detail on the selection of components for a SLIPS surface can be found in International Patent Application Nos. PCT/US2012/21928 and PCT/US2012/21929, both filed Jan. 19, 2012; International Patent Application No. PCT/US2012/63609, filed Nov. 5, 2012; U.S. Patent Application No. 61/555,957, filed Nov. 4, 2011, entitled Dynamic Slippery Surfaces; U.S. Patent Application No. 61/671,442, filed Jul. 13, 2012, entitled SELECTIVE WETTING AND TRANSPORT SURFACES; U.S. Patent Application No. 61/671,645, filed Jul. 13, 2012, entitled HIGH SURFACE AREA METAL OXIDE-BASED COATING FOR SLIPS; and U.S. Patent Application No. 61/673,705, filed Jul. 19, 2012, entitled MULTIFUNCTIONAL REPELLENT MATERIALS, the contents of which are hereby incorporated by reference in their entireties.

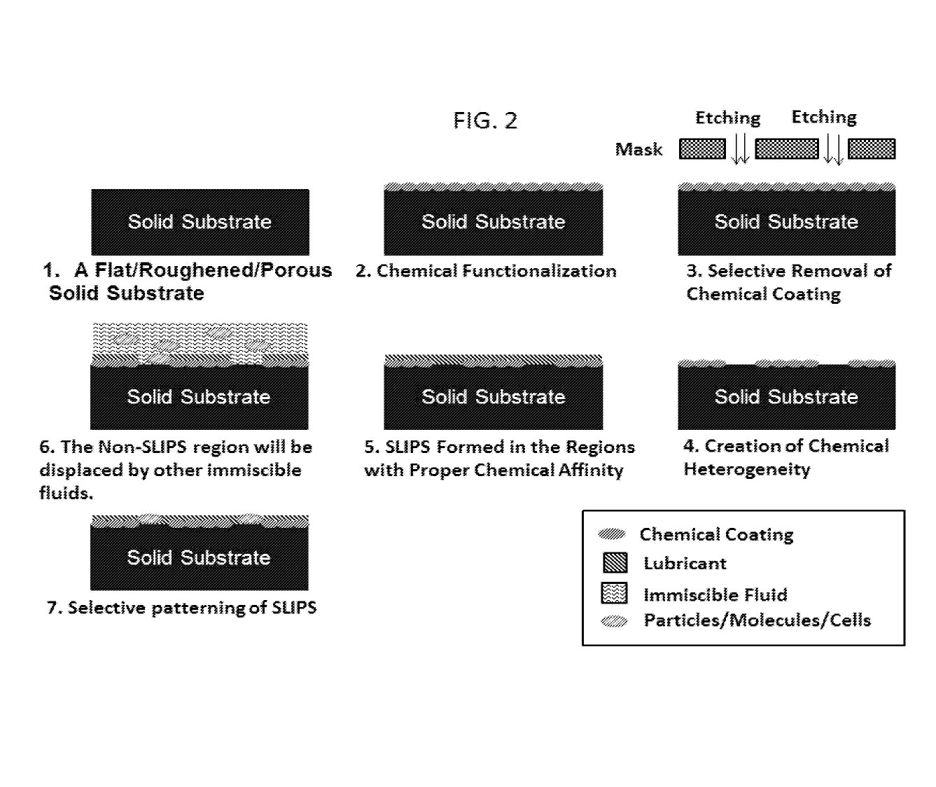

In some embodiments, a spatially heterogeneous pattern on a liquid-coated surface is created by first functionalizing a solid surface with spatially defined regions having different surface energies. Spatially defined regions are those which occupy less than the total area of a given surface of the substrate, that is, the surface energy properties of the surface varies laterally. Exemplary methods of creating such spatially defined regions include chemical functionalization with different surface functionalities, patterning with different materials and variation in surface roughness of the same material. Any other patterning methods that result in the localized changes of the surface properties are applicable. When a given lubricant is contacted with such a patterned surface, certain regions form a stable lubricant film owing to the matching in surface energies between the solid and lubricant (i.e. ΔE₁>0 and ΔE₂>0), whereas the rest of the regions remain unstable (i.e. ΔE₁<0 and/or ΔE₂<0). When a suitable immiscible liquid, which can have higher affinity to the surface in the unstable region, encounters the unstable lubricating region, it can displace the lubricant and remain trapped within the patterned region (see FIGS. 2-4).

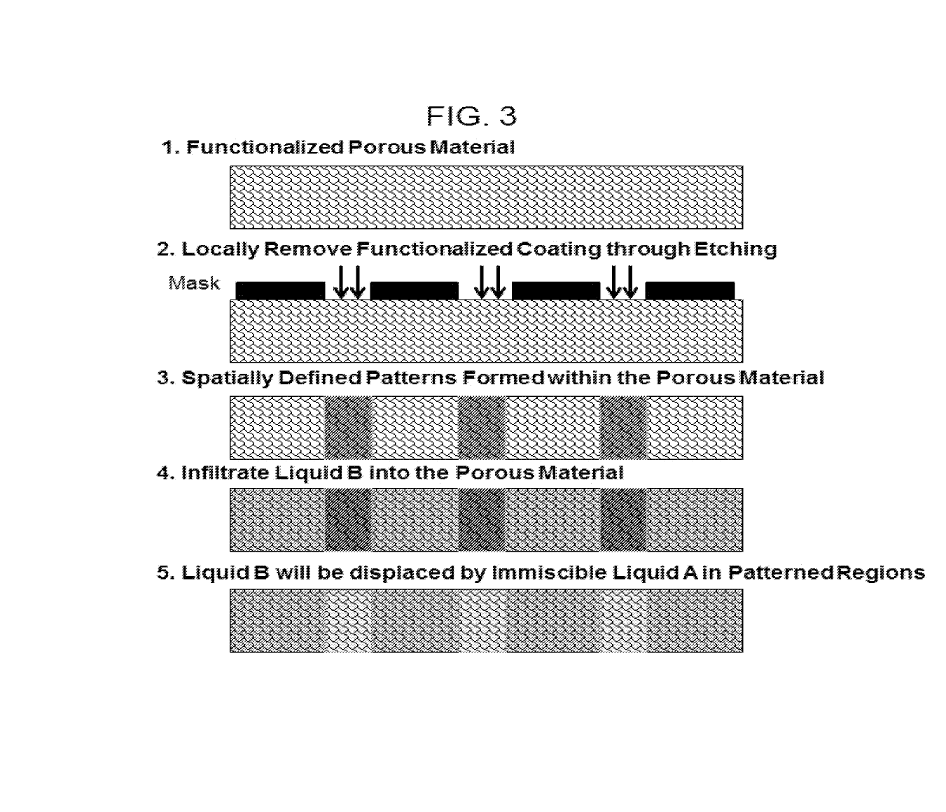

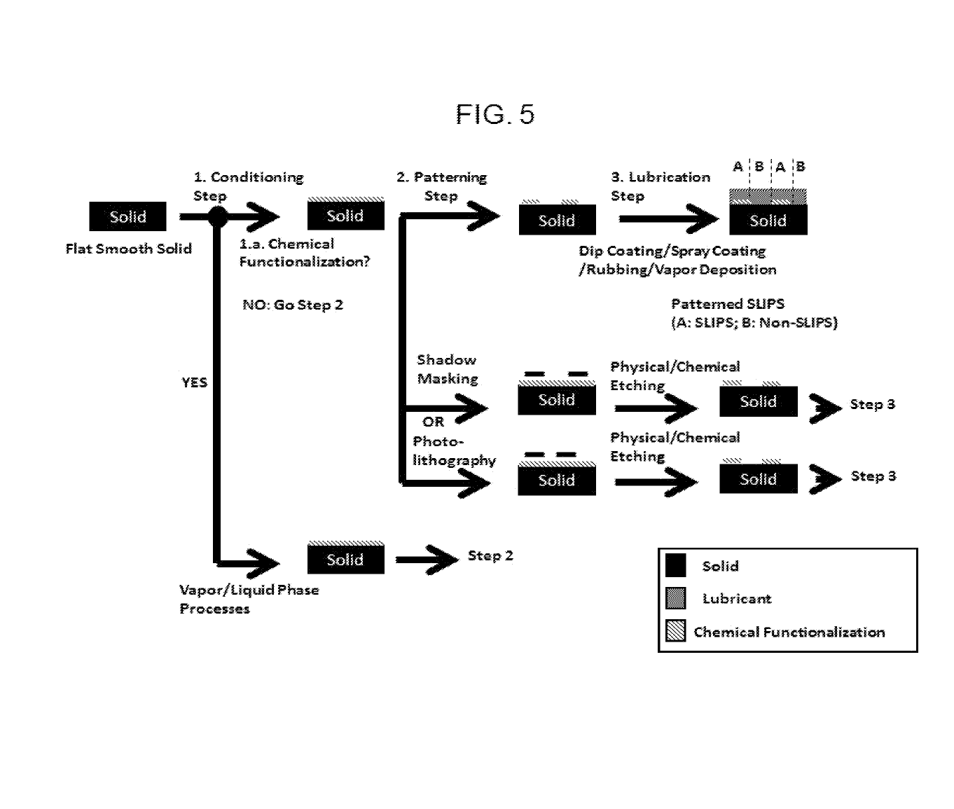

FIG. 3 shows the concept of patterning on slippery liquid-coated solid. In step 1, a functionalized porous material is provide. In some embodiments, the material is textured. In step 2, a mask is applied and functionalization is locally and selectively removed by etching. Step 3 shows spatially defined patterns formed within the porous material as a result of the etching in step 2. In step 4, Liquid B is infused into the porous material. As shown in step 4, Liquid B has affinity for the functionalized porous material, and a lesser degree of affinity for the areas where functionalization has been removed. In step 5, immiscible Liquid A is applied to the porous material. As shown in step 5, because Liquid A has a greater affinity for the areas where functionalization has been removed when compared to Liquid B, Liquid A displaces Liquid B and occupies these areas. As also shown in step 5, Liquid B remains in the functionalized porous areas, as Liquid A lacks sufficient affinity for the functionalized porous areas to displace Liquid B from this regions. The result of step 5 shows an embodiment of spatially heterogeneous patterned SLIPS. Potential applications include spatially defined patterning of cells for tissue engineering, mechanobiology, and single cell study, patterning of biological fluids, as well as high sensitivity biological sensors. Other applications include microfluidics, controlled placement of molecules or materials without cross-contamination, etc. In some embodiments, the material provided in step 1 can be smooth, and functionalization can be added locally to provide a spatially heterogeneous surface (see FIG. 5). In some embodiments, functionalization can be removed by providing a hole, pore, aperture, channel, well, void or perforation through the solid, such that the material defines holes, pores, apertures, voids or perforations. In some embodiments, Liquid B has a lesser degree of affinity for the holes, pores, apertures, channels, wells, voids or perforations than for the functionalized porous material and is displaced in these areas by Liquid A, which has a higher affinity for the hole, pore, aperture, channel, well, void or perforation as compared to Liquid B.

In certain embodiments, the liquid-coated substrate comprises patterning through at least a portion of the vertical thickness of the substrate. In some embodiments, the liquid-coated substrate comprises a heterogeneous surface pattern which is disposed through at least a portion of the thickness of the substrate. In certain embodiments, the second region is capable of wicking a wicking liquid into or through the substrate. In further embodiments, the liquid-coated substrate comprises a thickness, at least an upper layer comprising less than the total thickness of the substrate and a lower layer comprising less than the total thickness of the substrate; wherein said upper layer comprises a first side of said substrate; at least a portion of said first side having surface characteristics that provides a stable liquid film with a first lubricating liquid; and a first lubricating liquid disposed over said at least a portion of said at least a portion of said first side.

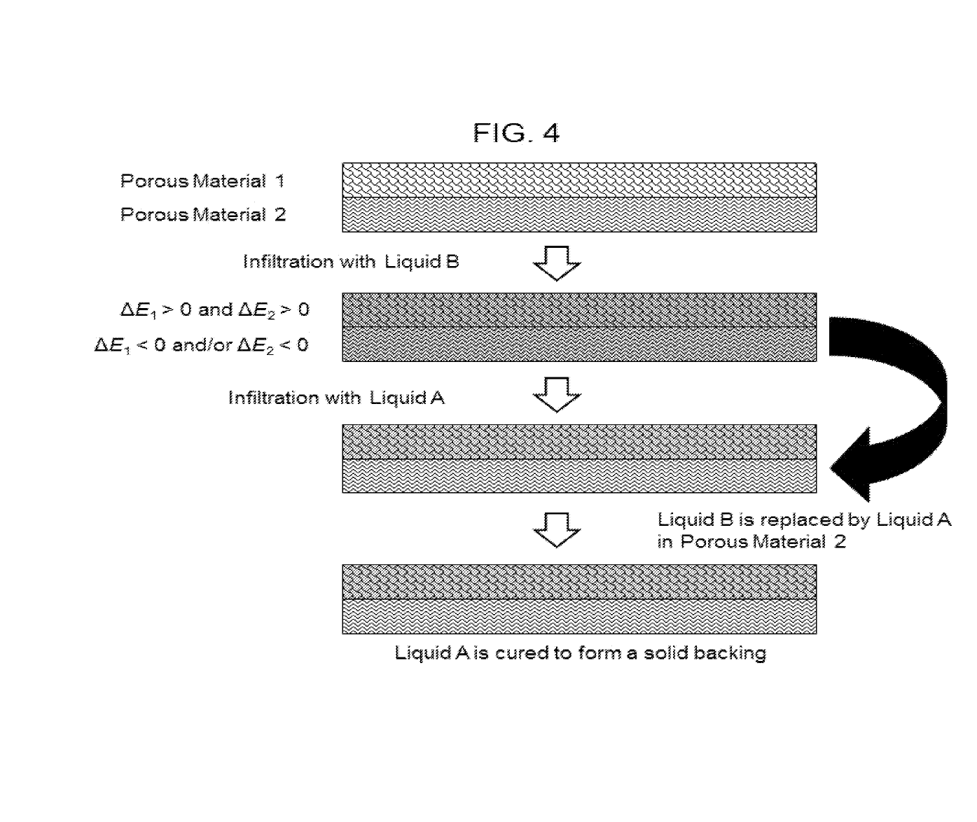

In some embodiments, vertically patterned liquid-coated substrates are formed by adhering two different materials together to form a single substrate, such that the substrate comprises at least a top layer and a bottom layer which display different surface characteristics. For example, as shown in FIG. 4, Porous Material 1 and Porous Material 2 may be joined together such that the formed substrate has an upper layer comprising Porous Material 1 which has an affinity for Liquid B and a lower layer comprising Porous Material 2 which has an affinity for Liquid A. In other embodiments, a substrate can be formed of a single material having a thickness with a particular surface characteristic, wherein a second surface characteristic is selectively introduced onto one side of the substrate. In some embodiments, the second surface characteristic is introduced by additive or subtractive processes. For example, in some embodiments a substrate having a first surface characteristic is chemically or physically etched selectively on one side through less than the total thickness of the substrate to provide a single substrate having different surface characteristics on opposing sides. In other embodiments, a substrate having a first surface characteristic is physically or chemically functionalized selectively on one side to provide a single substrate having different surface characteristics on opposing sides.

In certain embodiments, heterogeneous topographies or spatially-defined patterns of selective wettability on a liquid-coated or liquid-infiltrated solid substrate (SLIPS) impart different functionalities to different regions of the articles. The regions that allow selective wetting (e.g., of an aqueous phase) can allow, by way of non-limiting example, local culture of cells, or transport of liquid through a SLIPS layer for sensing or drainage functions. The combination of these ultra-low adhesion and selective wetting (or wicking) properties can be used for applications for patterning of biological and non-biological substances, printing of characters, creating liquid adhesives, or permeable/non-permeable solid support, or for the design of bandage or ‘breathing skin layer’ biomedical materials

In certain embodiments, a non-adhesive article capable of absorbent wicking is disclosed which comprises a wound dressing, or bandage, specifically for protecting wounds, burns or skin trauma from exposure and infection. The absorbent wicking allows drainage of the exudate fluid from the wound (to an absorbent layer), and the non-adhesive surface prevents adhesion of the wound tissue to the bandage. The SLIPS lubricant can be highly soluble and permeable to oxygen, to allow a high flux of oxygen to the wound tissue surface, to allow improved wound repair. In addition, the bandage can provide one or more of the following properties. The bandage can be selected from materials to provide oxygen permeability to the wound surface. The non-adhesive article capable of absorbent wicking can be selected from materials to provide absorbance of wound exudate fluid, blood, pus, etc. It can be non-adhesive to the underlying tissue, but include an adhesive strip for securing to healthy tissue.

In some embodiments, the first region of the substrate is chemically functionalized to provide at least in part a surface energy that provides a stable or meta-stable liquid film with the lubricating liquid. In further embodiments, the second region of the substrate is chemically functionalized to provide at least in part a surface energy that provides a stable film with a second liquid. The exact surface functionalization is substrate specific. For example, alkylphosphonates can be used to functionalize alumina, while silanes are good for surfaces that contain or that were pretreated to contain hydroxyl groups. In some embodiments, chemical functionalization is accomplished with silanes, and can be for example chlorosilanes, ethoxysilanes, methoxysilanes with a range of hydrocarbon and fluorocarbon chains. A common surface treatment can be hexamethyldisilane (HMDS) or heptadecafluoro-1,1,2,2-tetrahydrodecyltrichlorosilane. In some embodiments, chemical functionalization is accomplished by vapor deposition.

In some embodiments, before the wetting layer-coated substrate is exposed to the second liquid, the substrate is completely wetted by the first lubricating liquid, notwithstanding the lesser affinity of the first lubricating liquid for the second region.

In some embodiments, the article is functionalized on a first surface with a roughened, structured or porous surface infused with a lubricating liquid that provides an ultrasmooth, slippery surface (i.e. a stable liquid film). The roughened, structured or porous surface can be a functionalized or modified portion of the first surface, or it can be a layer that is applied to the article. By way of example, the roughened, structured or porous surface can be a porous sheet applied onto the article. In other embodiments, the roughened, structured or porous surface can be a molded micro- or nanostructure, or it can be a roughened surface obtained by particle spraying, sandblasting, embossing, imprinting, electrodeposition, or etching the surface of the substrate. In some embodiments, the roughened surface is further chemically or physically functionalized, when needed, to provide the high affinity to a lubricant that allows the lubricant to be stably attached to the surface. In some embodiments, the layer of lubricating liquid is thin and relatively immobilized on the roughened or porous surface, that is, the interaction between the substrate and the lubricating liquid is sufficiently strong enough to prevent the free flow of the liquid over and from the surface (i.e. a stable liquid film is formed). In some embodiments, volume of lubricating liquid is present at a level sufficient to just cover the highest projections of the roughened surface.

In certain embodiments, the present disclosure describes solid matrices comprising a SLIPS surface and a liquid adhesive surface provided by selective displacement of lubricant. In certain embodiments, a 2-layer porous solid composed of two different types of material is provided, wherein one layer has matching surface energy to Liquid B (but does not have matching surface energy to Liquid A), while the other layer has matching surface energy to Liquid A (but does not having matching surface energy to Liquid A). Accordingly, in certain embodiments, the material can be treated with a non-adhesive Liquid B, while an adhesive Liquid A displaces the portion of Liquid B in contact with the layer of the material which has matching surface energy to Liquid A. This embodiment therefore provides a 2-layer material which can adhere to a secondary substrate on one side, while providing an outer SLIPS surface. In certain embodiments, Liquid A is chosen such that it can polymerize to form permanent bonding with the secondary solid substrate.

In certain embodiments, the upper porous region of the porous substrate is chemically functionalized to provide at least in part a surface energy that provides a stable liquid film with the lubricating liquid. In further embodiments, the lower porous region of the porous substrate is chemically functionalized to provide at least in part a surface energy that provides an unstable film with the lubricating liquid. In some embodiments, chemical functionalization is accomplished with heptadecafluoro-1,1,2,2-tetrahydrodecyltrichlorosilane. In some embodiments, chemical functionalization is accomplished by vapor deposition.

In certain embodiments, the upper porous layer of the substrate is hydrophobic or omniphobic and the lower porous layer of the substrate is hydrophilic.

### Roughened Surface

As used herein, the term “roughened surface” includes both the surface of a three-dimensionally porous material as well as a solid surface having certain topographies, whether they have regular, quasi-regular, or random patterns.

In certain embodiments, the roughened surface may have a roughness factor, R, greater than 1, where the roughness factor is defined as the ratio between the real surface area and the projected surface area. For complete wetting of Liquid B to occur, it is desirable to have the roughness factor of the roughened surface to be greater or equal to that defined by the Wenzel relationship (i.e. R≧1/cos θ, where θ is the contact angle of Liquid B on a flat solid surface). For example, if Liquid B has a contact angle of 50° on a flat surface of a specific material, it is desirable for the corresponding roughened surface to have a roughness factor greater than ˜1.5.

In certain embodiments, the presence of a roughened surface can promote wetting and spreading of Liquid B over the roughened surface. In certain embodiments, the roughened surface can be manufactured from any suitable materials. For example, the roughened surface can be manufactured from polymers (e.g., epoxy, polycarbonate, polyester, nylon, Teflon, etc.), metals (e.g., tungsten, aluminum, stainless steel, copper, zinc, and titanium),), sapphire, glass, carbon in different forms (such as diamond, graphite, black carbon, etc.), ceramics (e.g., alumina, silica), and the like. For example, fluoropolymers such as polytetrafluoroethylene (PTFE), polyvinylfluoride, polyvinylidene fluoride, fluorinated ethylene propylene, and the like can be utilized. In addition, roughened surfaces can be made from materials that have functional properties such as conductive/non-conductive, and magnetic/non-magnetic, elastic/non-elastic, light-sensitive/non-light-sensitive materials. A broad range of functional materials can make SLIPS.

In certain embodiments, the roughened surface may be the porous surface layer of a substrate with arbitrary shapes and thickness. The porous surface can be any suitable porous network having a sufficient thickness to stabilize Liquid B, such as a thickness from above 100 nm, or the effective range of intermolecular force felt by the liquid from the solid material. Below 100 nm thick, the liquid may start to lose its liquid property. The substrates can be considerably thicker, however, such as metal sheets and pipes. The porous surface can have any suitable pore sizes to stabilize the Liquid B, such as from about 10 nm to about 2 mm. Such a roughened surface can also be generated by creating surface patterns on a solid support of indefinite thickness.

Many porous materials are commercially available, or can be made by a number of well-established manufacturing techniques. For example, PTFE filter materials having a randomly arranged three-dimensionally interconnected network of holes and PTFE fibrils are commercially available.

Porous materials can be produced through direct modification of an aluminum substrate. For example, aluminum can be sandblasted to create hierarchical roughness, followed by ultrasonic cleaning in acetone and finally boiling in distilled water. The resulting Boehmite surface can then be chemically modified by a number of methods known in the art. In some embodiments, the substrate can be partially or selectively functionalized in this manner, resulting in a substrate with both roughened and non-roughened regions. Production of porous materials produced through direct modification of an aluminum or other metal substrate are discussed in further detail in U.S. Patent Application No. 61/671,645, filed Jul. 13, 2012, titled HIGH SURFACE AREA METAL OXIDE-BASED COATING FOR SLIPS and co-pending International Application entitled SLIPS SURFACE BASED ON METAL-CONTAINING COMPOUND, the contents of which are hereby incorporated by reference in their entirety.

SLIPS surfaces can also be made from transparent sol-gel alumina-based Boehmite coatings. For example, an alumina sol-gel precursor can be prepared from aluminum tri-tert-butoxide, ethylacetoacetate, 2-propanol and water and then spin or spray coated on a substrate, dried and treated with distilled water to provide a thin Boehmite coating which can be subsequently used to form a SLIPS surface. Sol-gel coatings can be applied to a variety of substrates, such as polysulfone, poly(methyl methacrylate) (PMMA), polycarbonate, polystyrene, polyurethane, epoxy, polyolefins, polyvinylchloride (PVC), polyethylene terephthalate (PET), glass and stainless steel. Sol-gel coatings can be applied in a variety of thicknesses, for example, 10 nm, 50 nm, 100 nm, 110 nm, 150 nm, 200 nm, 250 nm, 300 nm, 400 nm and 500 nm. In some embodiments, sol-gel derived Boehmite coatings are transparent and/or anti-reflective.

Patterned roughened surfaces can also be obtained in a variety of well-established techniques. In some embodiments, a substrate is patterned with non-uniform chemical functionalization of a structurally uniform substrate.

In some embodiments, a substrate is functionalized using select colloidal deposition. Colloidal deposition can be used to prepare thin films on substrates that give rise to rough surfaces with effective omniphobic behavior without affecting the optical properties of the substrate. Colloidal surface coatings comprise inverse opal structures, either in form of monolayers (2D) or 3D arrangements of colloids that are backfilled with silica precursor materials. The colloids can be removed to give rise to an inverse porous network of silica. The surface functionalities of the silica can be tuned according to the desired application. Specifically, it can be made hydrophilic, hydrophobic or fluorophilic by silanization reactions. Using fluoro-silanization, a stable SLIPS state is created by addition of fluorinated lubricants. This addition induces omniphobic behavior to the substrate: liquids or dispersions are effectively repelled from the substrate and do not leave traces. Colloidal deposition can be selectively applied by photolithographic methods using a structured substrate, followed by selective silanization of the exposed regions of the surface. Full transparency of the coating can be achieved by three methods: 1) applying colloidal monolayers as templates with colloid sizes smaller than 400 nm; 2) applying colloidal monolayer with a high degree of damage to thin out the optical thickness of the film; 3) applying 3D opal films composed of two or more different colloids with sizes chosen in a way to prevent the formation of a regularly ordered crystal. Functionalization of a substrate using colloidal deposition is discussed in further detail in co-pending International Patent Application entitled SLIPPERY LIQUID-INFUSED POROUS SURFACES HAVING IMPROVED STABILITY, filed on even date herewith, the contents of which are hereby incorporated by reference in their entirety.

A schematic for a series of processes of making patterned SLIPS from a flat smooth solid is shown in FIG. 5. The process includes an optional conditioning step, a patterning step and a lubrication step. Chemical functionalization can be used to provide surface energies used to satisfy SLIPS conditions. In some embodiments, a flat smooth solid is chemically functionalized by vapor or liquid phase processes, patterned by either screening, printing, shadow masking or photolithography, with the resulting solid being physically or chemically etched. The etched solid is then either dip coated, spray coated, rubbed or subjected to vapor deposition prior to exposure to a lubricating liquid, resulting in patterned SLIPS. In other embodiments, a flat solid is patterned without chemical functionalization by either screening, printing, shadow masking or photolithography, with the resulting solid being physically or chemically etched. The etched solid is then either dip coated, spray coated, rubbed or subjected to vapor deposition prior to exposure to a lubricating liquid, resulting in patterned SLIPS.

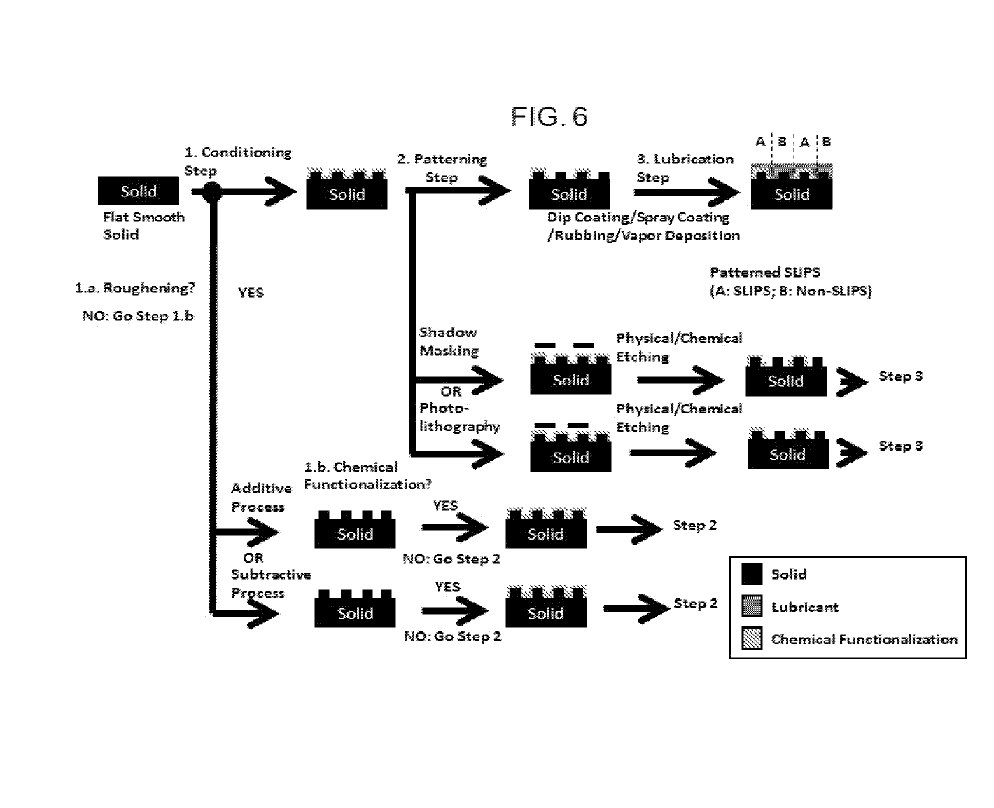

In some embodiments, it is desired to provide regions of varying characteristics that extend not only along the surface of the article, but also into the bulk or thickness of the layer. A schematic for a series of processes of making 2.5D patterned SLIPS, e.g., a surface pattern that is carried out through at least a portion of the thickness of the layer of an initially smooth substrate is shown in FIG. 6, which is processed to create roughness. In some embodiments, a solid is roughened either by additive or subtractive processes. The resulting roughened solids are either chemically functionalized and then patterned, or directly patterned by either printing, shadow masking or photolithography. The patterned solid is then either physically or chemically etched. The resulting solid can then be dip coated, spray coated, rubbed or undergo vapor deposition. Lastly, a lubricating liquid is added to form a patterned SLIPS surface. In other embodiments, a pre-roughened solid is patterned by either shadow masking or photolithography. The patterned solid is then either physically or chemically etched. The resulting solid can then be dip coated, spray coated, rubbed or undergo vapor deposition. Lastly, a lubricating liquid is added to form a patterned SLIPS surface.

Roughening processes known in the art may be used. Exemplary processes for roughening include application of liquid phase material (paint or ink, spray, spin, dip, air brush, screen printing, inkjet printing);—deposition or reaction of gas phase material (CVD, plasma, corona. ALD, PVD),—sputtering or evaporation of metal or metal oxide, composite phase material deposition (particle+binder), electrodeposition or other solution phase growth of material (conducting polymer, electroplated metal, electrophoretic deposition of particles, surface-initiated polymerization, mineralization), gas phase growth of material (nanofibers), multiple layer deposition (repeated coating, layer-by-layer deposition), self-assembly of precursor material (minerals, small molecules, biomolecules, polymers, nanoparticles, colloids), or growth of layers by oxidation-transfer coating and printing (contact printing, pattern transfer).

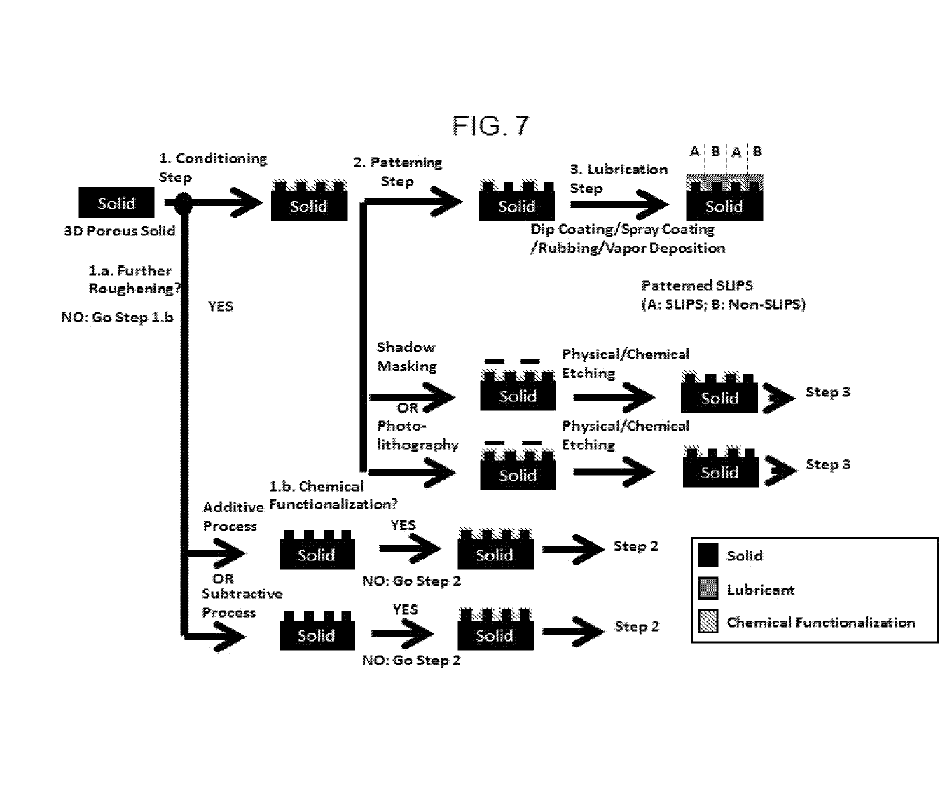

A schematic for a series of processes of making 3D patterned SLIPS is shown in FIG. 7. In contrast to FIG. 6, the starting material in FIG. 7 is porous, and can be used to create a SLIPS surface. In some embodiments, a solid is roughened either by additive or subtractive processes. The resulting roughened solids are either chemically functionalized and then patterned, or directly patterned by either printing, screening, shadow masking or photolithography. The patterned solid is then either physically or chemically etched. The resulting solid can then be dip coated, spray coated, rubbed or undergo vapor deposition. Lastly, a lubricating liquid is added to form a patterned SLIPS surface. In other embodiments, a pre-roughened solid is patterned by either shadow masking or photolithography. The patterned solid is then either physically or chemically etched. The resulting solid can then be dip coated, spray coated, rubbed or undergo vapor deposition. Lastly, a lubricating liquid is added to form a patterned SLIPS surface.

In some embodiments, the solid used to create SLIPS has chemical affinity for a lubricating liquid. In these embodiments, when a lubricating liquid is applied to the substrate, the lubricating liquid will preferentially wet portions of the substrate which have not been functionalized or roughened, causing these regions to also act as SLIPS.

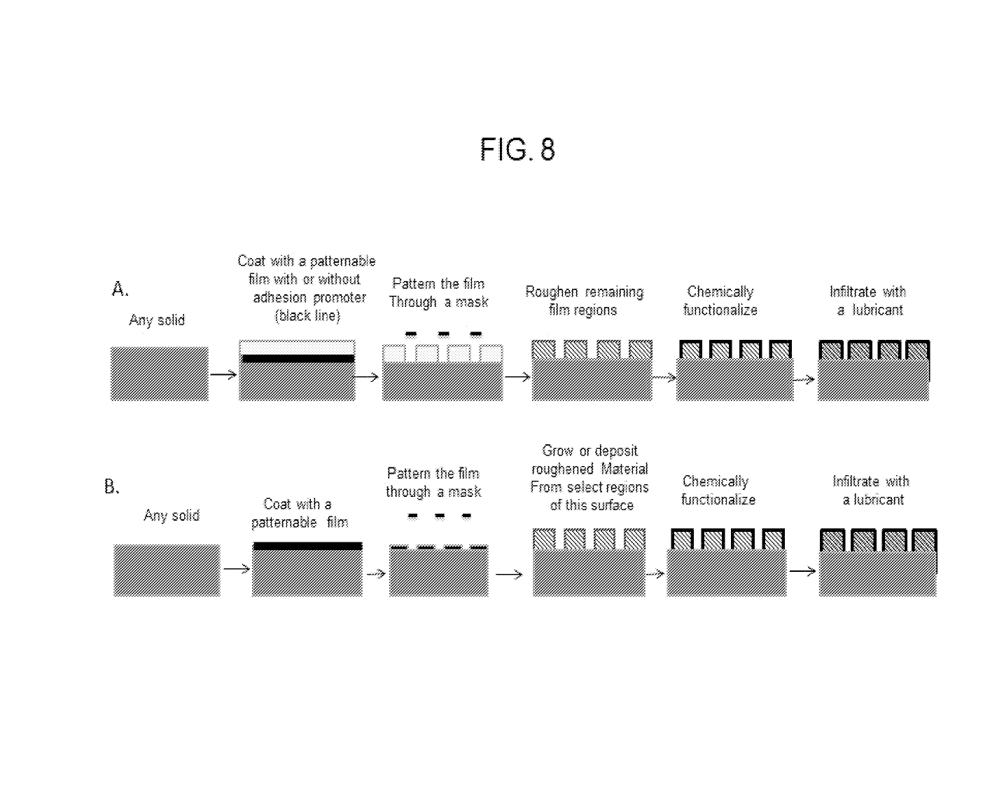

In some embodiments, a SLIPS surface is applied as a coating to an article, as depicted in FIG. 8A. A solid is coated with a patternable with our without an adhesion promoter, and the film is patterned through a mask. The remaining film regions are then roughened, chemically functionalized, and a lubricant is added to infiltrate the functionalized areas. In some embodiments, this scheme depicts the formation of a patterned Boehmite substrate.

In some embodiments, a SLIPS article is generated from a solid as depicted in FIG. 8B. A solid is coated with a patternable with or without an adhesion promoter, and the film is patterned through a mask. An adhesion propomoter may be used to create a strong adhesion of the patternable surface to the underlying based substrate. Roughened materials are then grown or deposited on selected regions of the surface. The roughened regions are then chemically functionalized, and a lubricant is added to infiltrate the functionalized areas. In some embodiments, this scheme depicts selective colloidal disposition or localized growth of nanofibers on metalized regions of the substrate. In some embodiments, the nanofibers comprise polypyrrole.

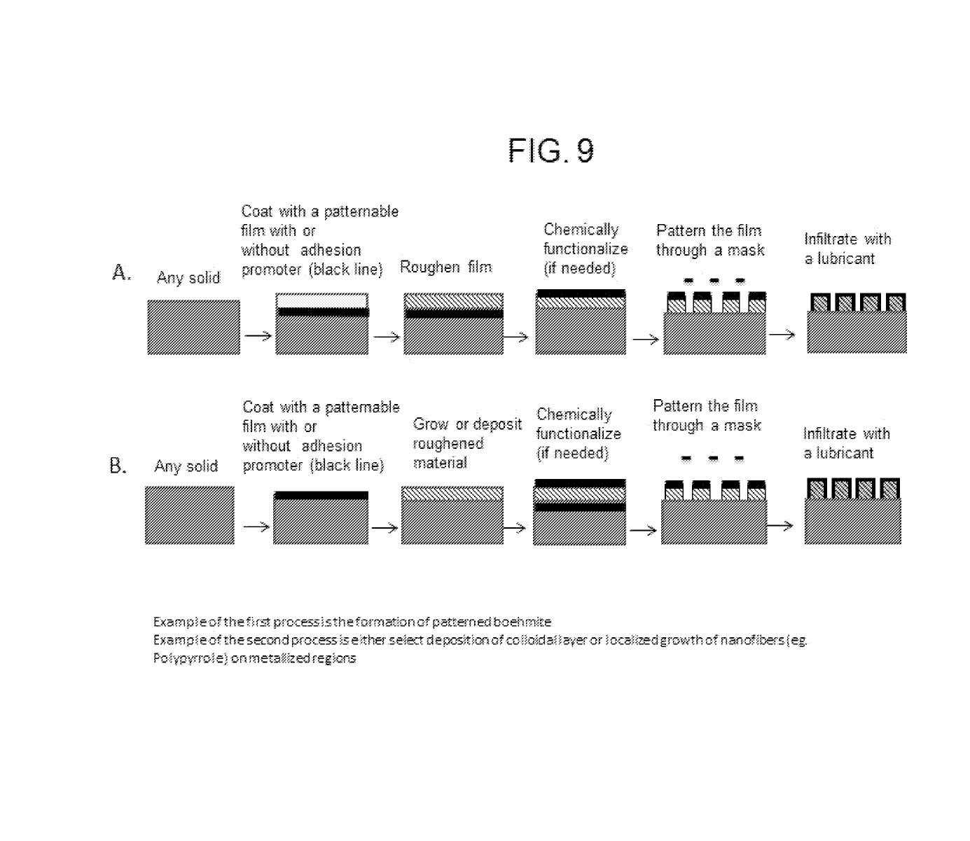

In some embodiments, a SLIPS article is generated using the coating and pattering process depicted in FIG. 9A. A solid is coated with a patternable material with our without an adhesion promoter, and the film is roughened. The film is then optionally chemically functionalized, and the film is patterned through a mask and a lubricant is added to infiltrate the functionalized areas. In some embodiments, this scheme depicts the formation of a patterned Boehmite substrate.

In some embodiments, a SLIPS article is generated from a solid as depicted in FIG. 9B. A solid is coated with a patternable with our without an adhesion promoter, and roughened material is grown or deposited on the substrate. Roughened materials are then grown or deposited on the substrate. The roughened regions are then optionally chemically functionalized, the film is patterned through a mask, and a lubricant is added to infiltrate the functionalized areas. In some embodiments, this scheme depicts selective colloidal disposition or localized growth of nanofibers on metalized regions of the substrate. In some embodiments, the nanofibers comprise polypyrrole.

### Object A

### Physical Size of Object A Relative to Its Capillary Length

In certain embodiments, Object A may slide off from SLIPS by gravity when the surface is tilted at an angle with respect to the horizontal, given that the size of Object A, either in liquid form or in solidified form, is larger than a characteristic size. Specifically, the effect of gravity on Object A may be more dominant when its size is much larger than the capillary length of Liquid A. Specifically, capillary length is a characteristic length scale that quantifies the dominance of body force over surface force on an object, which can be quantitatively expressed as (γ/pg)^{1/2}, where γ, ρ, and g are surface tension and density of the liquid, and gravity, respectively. For example, size of Solid A or of Liquid A may be at least 3 times larger than the capillary length of Liquid A.

As noted previously, a wide range of materials can be repelled by the slippery surfaces of the present disclosure. For example, Object A can include polar and non-polar Liquids A and their solidified forms, such as hydrocarbons and their mixtures (e.g., from pentane up to hexadecane and mineral oil, paraffinic extra light crude oil; paraffinic light crude oil; paraffinic light-medium crude oil; paraffinic-naphthenic medium crude oil; naphthenic medium-heavy crude oil; aromatic-intermediate medium-heavy crude oil, aromatic-naphthenic heavy crude oil, aromatic-asphaltic crude oil, etc.), ketones (e.g., acetone, etc.), alcohols (e.g., methanol, ethanol, isopropanol, dipropylene glycol, ethylene glycol, and glycerol, etc.), water (with a broad range of salinity. e.g., sodium chloride from 0 to 6.1 M; potassium chloride from 0 to 4.6 M, etc.), acids (e.g., concentrated hydrofluoric acid, hydrochloric acid, nitric acid, etc.) and bases (e.g., potassium hydroxide, sodium hydroxide, etc.), wine, soy sauce and the like, ketchup and the like, olive oils and the like, grease, soap water, surfactant solutions, and frost or and ice, etc. Object A can include biological objects, such as insects, blood, small animals, protozoa, bacteria (or bacterial biofilm), viruses, fungi, bodily fluids and tissues, proteins and the like. Object A can include solid particles (e.g., dust, smog, dirt, etc.) suspended in liquid (e.g., rain, water, dew, etc.). Object A can include non-biological objects, such as dust, colloidal suspensions, spray paints, fingerprints, food items, common household items, and the like. Object A can include adhesives and adhesive films. The list is intended to be exemplary and the slippery surfaces of the present disclosure are envisioned to successfully repel numerous other types of materials.

In certain embodiments, more than one different Object A can be repelled. In certain embodiments, the combination of two or more Object A may together be more readily repelled as compared to just one Object A.

### Liquid B

Liquid B (alternatively referred to as the “lubricant” or “wetting liquid” through the specification) can be selected from a number of different materials, and is chemically inert with respect to the solid surface and Object A. Liquid B flows readily into the surface recesses of the roughened surface and generally possesses the ability to form an ultra-smooth surface when provided over the roughened surface. In certain embodiments, Liquid B possesses the ability to form a substantially molecularly flat surface when provided over a roughened surface. The liquid can be either a pure liquid, a mixture of liquids (solution), or a complex fluid (i.e., a liquid+solid components).

In certain other embodiments, Liquid B possesses the ability to form a substantially molecularly or even atomically flat surface when provided over a roughened surface. This surface can make either a flat overlayer coating the entire structured surface, or follow the topography of the surface structures conformally.

In other embodiments Liquid B follows the topography of the structured surface and forms a conformal smooth coating (e.g., instead of forming a smooth layer that overcoats all the textures). For example Liquid B may follow the topography of the structured surface if the thickness of the layer is less than the height of the textures. While a smooth layer that overcoats all the textures provides the best performance, conformal smooth lubricant coating, which follows the topography of the structured surface and can arise from the diminished lubricant layer, still shows significantly better performance than the underlying substrate that was not infused with Liquid B.

### Materials for Liquid B

Liquid B can be selected from a number of different liquids and the mixtures thereof. For example, perfluorinated hydrocarbons, organosilicone compound (e.g., silicone elastomer), or fluorinated silicones and the like can be utilized. In particular, the tertiary perfluoroalkylamines (such as perfluorotri-n-pentylamine. FC-70 by 3M, perfluorotri-n-butylamine FC-40, etc), perfluorodecalin, perflubron, perfluoroalkylsulfides and perfluoroalkylsulfoxides, perfluoroalkylethers, perfluorocycloethers (like FC-77) and perfluoropolyethers (such as Krytox™ family of lubricants by DuPont), perfluoroalkylphosphines and perfluoroalkylphosphineoxides as well as their mixtures can be used for these applications, as well as their mixtures with perfluorocarbons and any and all members of the classes mentioned. In addition, long-chain perfluorinated carboxylic acids (e.g., perfluorooctadecanoic acid and other homologues, including diacids, triacids, polyacids, and their hydroxy-substituted derivatives), fluorinated phosphonic and sulfonic acids, fluorinated silanes, alcohols, and combinations thereof can be used as Liquid B. The perfluoroalkyl group in these compounds could be linear, branched, or cyclic and some or all linear, branched, or cyclic groups can be only partially fluorinated. In addition, Liquid B can be selected from a number of different biocompatiable or food-compatible liquids, including but not limited to water, aqueous solutions, olive oil, canola oil, coconut oil, corn oil, rice bran oil, cottonseed oil, grape seed oil, hemp oil, mustard oil, palm oil, peanut oil, pumpkin seed oil, safflower oil, sesame oil, soybean oil, sunflower oil, tea seed oil, walnut oil, and a mixtures of any of the above oils. In addition, water or other aqueous fluids can be used for selective patterning that involves hydrophobic/hydrophilic pattern or and oils for selective patterning that involves olcophobic/olcophilic pattern.

### Density of Liquid B

In certain embodiments, Liquid B has a high density. For example, Liquid B has a density that is more than 1.0 g/cm³, 1.6 g/cm³, or even 1.9 g/cm³. In certain embodiments, the density of Liquid B is greater than that of Object A to enhance liquid repellency. High density fluids reduce the tendency of any impacting liquid to ‘sink’ below the surface of Liquid B and to become entrained therein. For Object A that is smaller than its capillary length (assume Object A is in liquid form), it is possible that the Liquid B has a density lower than that of the Object A, where the SLIPS formed by Liquid B can remain functional.

### Solidification Temperature

In certain embodiments, Liquid B has a low freezing temperature, such as less than −5° C., −25° C. or even less than −80° C. Having a low freezing temperature will allow Liquid B to maintain its slippery behavior at reduced temperatures and to repel a variety of liquids or solidified fluids, such as ice and the like, for applications such as anti-icing surfaces.

### Evaporation Rate

In certain embodiments, Liquid B can have a low evaporation rate, such as less than 1 nm/s, less than 0.1 nm/s, or even less than 0.01 nm/s. Taking a typical thickness of Liquid B to be about 10 μm and an evaporation rate of about 0.01 nm/s, the surface can remain highly liquid-repellant for a long period of time without any refilling mechanisms.

In certain embodiments, the lifetime of the surface can be further extended by using a self-refilling mechanism.

### Viscosity of Liquid B

Experimentally, it is observed that Liquid A can become highly mobile on the surface of Liquid B when the kinematic viscosity of Liquid B is less than 1 cm²/s. Since liquid viscosity is a function of temperature (i.e., liquid viscosity reduces with increasing temperature), choosing the appropriate lubricant that operates at the aforementioned viscosity (i.e. <1 cm²/s) at specific temperature range is desirable. Particularly, various different commercially available Liquid B can be found at the specified viscosity, such as perfluorinated oils (e.g., 3M™ Fluorinert™ and DuPont™ Krytox® oils), at temperatures ranging from less than −80° C. to greater than 260° C. For example, the temperature dependence of liquid viscosity of DuPont Krytox oils is shown in the Table A as a specific example (note: data is provided by the manufacturer of DuPont Krytox oils).

### Film Thickness

Liquid B can be deposited to any desired thickness. Thickness of Liquid B on the order of the surface roughness peak-to-valley distance of the porous substrate provides good liquid-solid interaction between the substrate and Liquid B. When the solid substrate is tilted at a position normal to the horizontal plane, liquid layer with thickness below a characteristic length scale can maintain good adherence to the roughened surface, whereas liquid layers above the characteristic length can flow, creating flow lines (surface defects) and disrupting the flatness of the fluid surface. For example, non-limiting thicknesses for the fluid layer (as measured from the valleys of the roughened surface are on the order of 5-20 μm when the peak to valley height is ˜5 μm.

### Reactivity Between Liquid B and Roughened Surface

The roughened surface material can be selected to be chemically inert to Liquid B and to have good wetting properties with respect to Liquid B. In certain embodiments, Liquid B (and similarly Object A) may be non-reactive with the roughened surface. For example, the roughened surface and Liquid B (or Object A) can be chosen so that the roughened surface does not dissolve upon contact with Liquid B (or Object A). In particular, perfluorinated liquids (Liquid B) work exceptionally well to repel a broad range of polar and non-polar Liquids A and their solidified forms, such as hydrocarbons and their mixtures (e.g., from pentane up to hexadecane and mineral oil, paraffinic extra light crude oil; paraffinic light crude oil; paraffinic light-medium crude oil; paraffinic-naphthenic medium crude oil; naphthenic medium-heavy crude oil; aromatic-intermediate medium-heavy crude oil; aromatic-naphthenic heavy crude oil, aromatic-asphaltic crude oil, etc.), ketones (e.g., acetone, etc.), alcohols (e.g., methanol, ethanol, isopropanol, dipropylene glycol, ethylene glycol, and glycerol, etc.), water (with a broad range of salinity, e.g., sodium chloride from 0 to 6.1 M; potassium chloride from 0 to 4.6 M, etc.), acids (e.g., concentrated hydrofluoric acid, hydrochloric acid, nitric acid, etc.) and bases (e.g., potassium hydroxide, sodium hydroxide, etc.), soap water, detergent, surfactant-rich solutions, frost, and ice, etc.

### Wettability of Liquid B

In addition, the roughened surface topographies can be varied over a range of geometries and size scale to provide the desired interaction, e.g., wettability, with Liquid B. In certain embodiments, the micro/nanoscale topographies underneath the Liquid B can enhance the liquid-wicking property and the adherence of Liquid B to the roughened surface. As a result, the Liquid B can uniformly coat the roughened surface and get entrapped inside at any tilting angles.

### Combination of Object A and Liquid B

### Immiscibility

In certain embodiments. Object A (i.e., the test liquid) and Liquid B (i.e., the functional liquid layer) may be immiscible. For example, the enthalpy of mixing between Object A and Liquid B may be sufficiently high (e.g., water and oil) that they phase separate from each other when mixed together.

In certain embodiments, Liquid B can be selected such that Object A has a small or substantially no contact angle hysteresis. Liquid B of low viscosity (i.e., <1 cm²/s) tends to produce surfaces with low contact angle hysteresis. For example, contact angle hysteresis less than about 5°, 2.5°, 2°, or even less than 1° can be obtained. Low contact angle hysteresis encourages test Object A sliding at low tilt angles (e.g., <5°), further enhancing liquid repellant properties of the surface. The mechanics of SLIPS surfaces are discussed in International Patent Application Nos. PCT/US2012/21928 and PCT/US2012/21929, both filed Jan. 19, 2012, the contents of which are hereby incorporated by reference in their entireties.

### Applications

Numerous different applications for SLIPS can be envisioned where surface that repel a wide range of materials is desired. Some non-limiting exemplary applications are described below.

### Patterning of Biological and Non-Biological Substances

In one aspect of the present disclosure, a substrate is functionalized so as to comprise both SLIPS and non-SLIPS regions so as to allow for selective displacement of slippery liquid (Liquid A) by the materials selected to occupy these spaces (Object A), according to the exemplary methods described above. According to one or more embodiments, the selective wetting and fluid transport on the SLIPS surface can be used to selectively locate a second fluid (and any solutes or particles contained within that second fluid) at selected locations on the substrate surface. Exemplary embodiments of this aspect of the present disclosure are shown in FIG. 2. In one embodiment, a SLIPS surface is functionalized as previously described to provide regions having a strong affinity to the lubricating liquid so as to form an SLIPS surface and regions that have a lesser affinity to the lubricating liquid, e.g., the liquid layer is not stable, thereby providing a surface having, a spatially heterogeneous pattern on a liquid-coated surface. See (5.) of FIG. 2. The spatially heterogeneous SLIPS surface is exposed to a second liquid containing a solute (molecule) or particle/cell of interest such that the second liquid displaces the lubricating liquid on the substrate. See (6.) of FIG. 2. The resulting liquid layer includes the second liquid localized on non-SLIPS regions of the substrate. See (7.) in FIG. 2.

In some embodiments, Object A is particles, such that the particles are selectively located on only the non-SLIPS regions of the substrate. In further embodiments, Object A is molecules, such that the molecules are selectively located on only the non-SLIPS regions of the substrate. In still further embodiments, Object A is cells, such that the cells are selectively located on only the non-SLIPS regions of the substrate.

In some embodiments, these patterned substances are useful for patterning of cells for tissue engineering, mechanobiology and single cell study. For example, the spatially heterogeneous SLIPS surface can be used to create nanowells for biological screening. In other embodiments, the spatially heterogeneous SLIPS surface can be used to create nanowells for isolation and culturing of different cells. In further embodiments, the patterned substances are useful for patterning of biological fluids or high sensitivity biological sensors. In further embodiments, the patterned substances are useful for patterning biological fluids and selective adhesion in lab-on-a-chip devices.

### Patterned Absorbing Materials/Fluid Transport Devices

In another aspect of the present disclosure, a spatially heterogeneous SLIPS surface is used to provide a patterned absorbing material having non-stick characteristics. In some embodiments, the absorbing material comprises a non-adhesive surface to allow infiltration, wicking or active transport of another liquid phase through the SLIPS surface. In some embodiments, the non-adhesive surface serves as a conduit for transport of a liquid phase. In some embodiments, the non-adhesive surface is hydrophobic, and the liquid phase being wicked is aqueous.

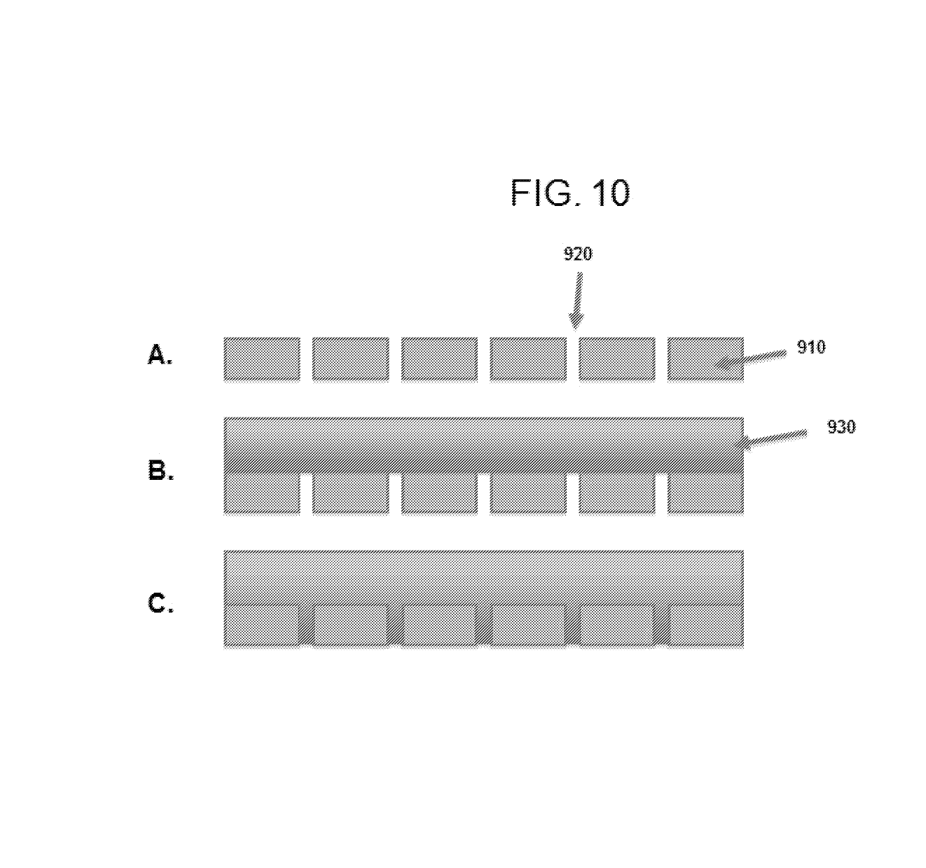

FIG. 10 shows a diagram of an exemplary embodiment of the present disclosure where a patterned SLIPS layer allows an aqueous phase to pass through a perforated SLIPS layer when placed into contact with the surface. FIG. 10A shows a substrate having porous or roughened regions 910 that can retain a lubricating layer and form a SLIPS surface. The substrate also includes holes, apertures, pores, aperture, channels, wells, voids or perforations 920 that pass through at least some thickness of the substrate. The holes, apertures, pores, channels, wells, voids or perforations are larger than any pores used in the SLIPS surface Large holes, apertures, pores, channels, wells, voids or perforations 920 in the SLIPS layer (i.e. the non-SLIPS regions) allow an infiltration of the aqueous phase 930, after overcoming an initial activation energy barrier to wetting within the holes, apertures, pores, channels, wells, voids or perforations, as illustrated in FIGS. 10B and 10C. In some embodiments, the aqueous phase passes through to the substrate or to a backing material (not shown).

In some embodiments, a patterned SLIPS layer is provided which incorporates an array of hydrophilic holes, apertures, pores, channels, wells, voids or perforations by patterned etching (such as plasma etching). In some embodiments, the hydrophilic holes, apertures, pores, channels, wells, voids or perforations become infiltrated with the infiltrating liquid phase (Liquid B, e.g., PFC or silicone), but become displaced by an aqueous phase (Liquid A) when coming in contact with an aqueous layer. In some embodiments, an aqueous phase such as blood, sweat, urine or exudate is able to wick through the SLIPS layer through holes, apertures, pores, channels, wells, voids or perforations 920.

FIG. 3 shows formation of patterned absorbing material in an exemplary embodiment of the present disclosure. In Step 1, a functionalized porous material is provided. In Step 2, the fictionalized porous material is etched locally to remove the chemical functionalization. Conversely, an unfunctionalized porous material can be provided in step 1 and functionalization can be introduced in selected areas in Step 2. The net effect of these two steps is to provide laterally heterogeneous regions in the substrate having different energetic interactin (affinity) with the infiltrating liquid phase (Liquid B, e.g., PFC or silicone) and the displacing liquid (Liquid A, e.g., aqueous phase), as illustrated in Step 3. In Step 4, the spatially defined porous material is infiltrated with a Liquid B. In this embodiment, the Liquid B and the non-patterned Porous Material have matching surface energy (i.e. ΔE₁>0 and ΔE₂>0) such that the liquid will be stably attached within the porous material as shown in Step 4. On the other hand, the Liquid B and patterned Porous Material do not have matching surface energy (i.e. ΔE₁<0 and/or ΔE₂<0). In Step 5, a Liquid A displaces Liquid B that is trapped within the unfunctionalized porous material. In this embodiment, the patterned region can provide drainage for Liquid A.

In some embodiments, the material is a wound dressing, bandage, sanitary pad or other medical (or surgical) non-bioresorbable tissue repair material. The non-sticking properties of SLIPS surfaces is used, for example, in medical applications such as wound or burn care, where it is desirable to prevent adhesion of sensitive skin. In some embodiments, the patterned absorbing materials comprises regions characterized by penetrability and non-penetrability of bodily fluids (such as blood, plasma, exudate, etc.). In certain embodiments, the regions characterized by penetrability of bodily fluids also provide air transfer to the wound or skin surface. In further embodiments, the patterned absorbing materials allow for draining of a wound without disturbing the general coating.

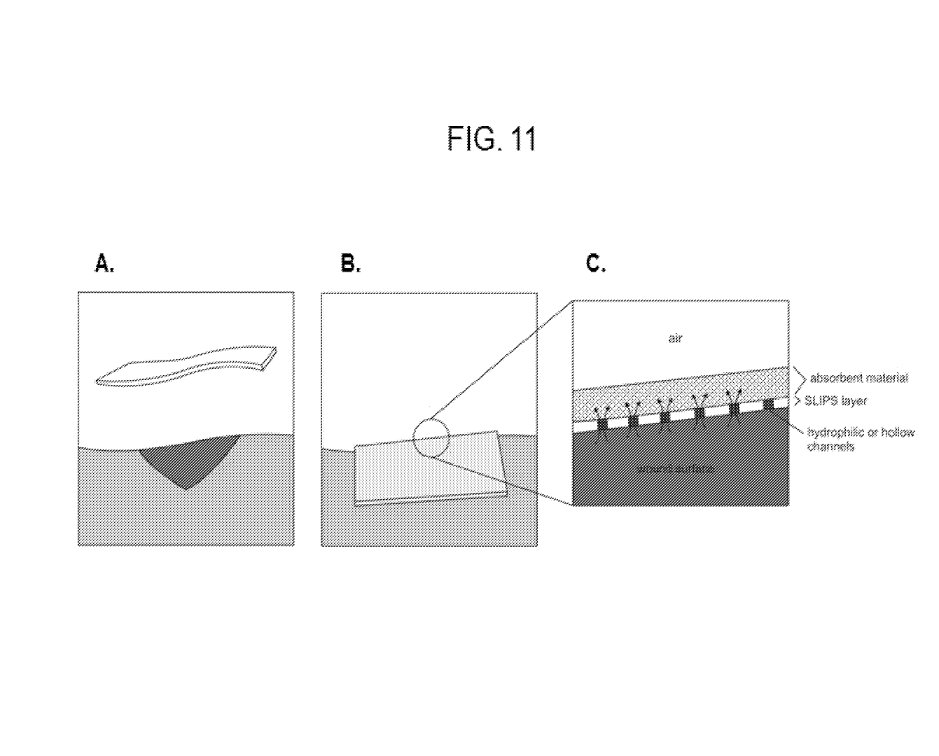

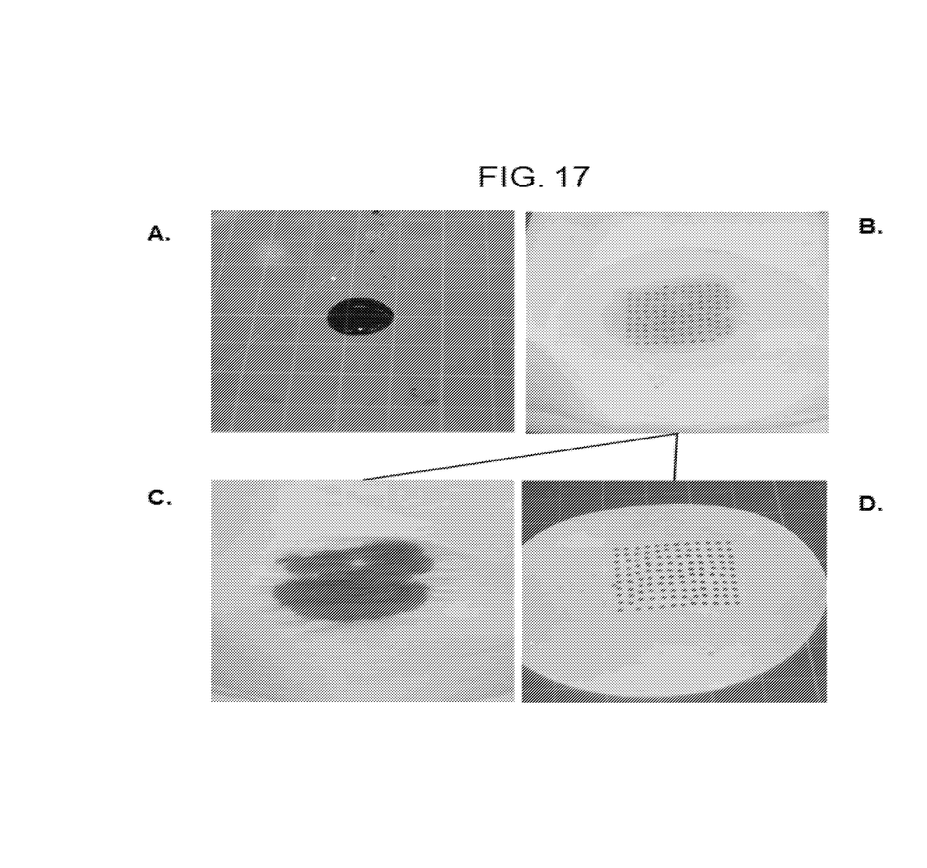

In embodiments where the patterned absorbing material is a wound dressing, bandage, sanitary pad, or other medical or surgical non-bioresorbable tissue repair material, these materials provide several advantages over prior solutions. These patterned absorbing materials remain adherent to the wound itself, including clotted blood, dried exudate, skin and regrown tissue. In addition, these patterned absorbing materials absorb exudate (such as blood, plasma, etc.) from the wound more efficiently than prior materials. Further, these materials provide the benefits of protecting the wound from infection, allowing efficient oxygen transfer to the wound, and effectively promotes tissue healing in an unexpectedly improved manner from prior materials. In some embodiments, the SLIPS material itself provides for high permeability of oxygen to the wound. FIGS. 11A-B shows an exemplary schematic where a SLIPS wound dressing or bandage is applied to a wound. FIG. 11C shows the removal of blood, plasma or exudate through the hydrophilic or hollow holes, apertures, pores, channels, wells, voids or perforations through the slips layer and to the absorbent backing. In some embodiments, the wound dressing or bandage further comprises oxygen-permeable lubricants which aid in wound and skin repair. In some embodiments, the lubricants are selected from perfluorocarbons and silicone oils. FIG. 17A-17D shows a series of images demonstrating a SLIPS membrane (ePTFE saturated with perfluorocarbon liquid) which contains an array of 1 mm diameter holes (which can be formed by laser cutting or a hole punch process) coupled with an absorbent backing layer (hydrophilic tissue) used as a bandage. This SLIPS bandage consists of a 2-layer laminate of the perforated SLIPS (ePTFE, and FC-70 lubricant) membrane layer, and the absorbent (hydrophilic ‘Kimwipe’ tissue) layer. An adhesive border can be included to provide adhesion to the skin (non-wound) surface. Further detail on forming adhesive layers/backings is found in U.S. Patent Application No. 61/671,442, filed Jul. 13, 2012, and International Patent Application entitled STRUCTURED FLEXIBLE SUPPORTS AND FILMS FOR LIQUID-INFUSED OMNIPHOBIC SURFACES, filed on even date herewith, the contents of which are incorporated by reference.

FIG. 17A shows an aqueous test fluid that is used to demonstrate absorption properties of the bandage. FIG. 17B shows the SLIPS membrane in contact with the test fluid, whereby the bandage immediately allows wicking through the SLIPS layer, to wet only the absorbing layer. FIG. 17C shows the separated absorbing material, which contains the test fluid, while FIG. 17D shows that the SLIPS layer has not absorbed any of the test fluid.

In some embodiments, a SLIPS layer is provided which can combine ultra-low adhesion (as an omniphobic surface) with an ability to wick or transport an aqueous phase through the layer. In some embodiments, a series of through-thickness holes, apertures, pores, channels, wells, voids or perforations are provided through the SLIPS surface, as a kind of SLIPS mesh or membrane. In some embodiments, the size of the pores, apertures, channels, wells, voids, holes or perforations is between about 0.5 mm to about 3 mm in diameter. In some embodiments, these through-thickness pores, apertures, channels, wells, voids, holes or perforations are large enough that the infiltrated liquid material (Liquid A. i.e. perfluorocarbon. PFC, or silicone) does not fill the pores, apertures, channels, wells, voids, holes or perforations, but does fill the holes of the porous layer. In these embodiments, an aqueous phase in contact with the surface would first have to overcome an energy barrier to wetting within the pores, apertures, channels, wells, voids, holes or perforations. In certain embodiments, this energy barrier depends on the size of the pores, apertures, channels, wells, voids, holes or perforations. In certain embodiments, because the inner wall of the pores, apertures, channels, wells, voids, holes or perforations comprises a SLIPS material, immiscible liquids pass through the pores, apertures, channels, wells, voids, holes or perforations more quickly than through unfunctionalized pores, apertures, channels, wells, voids, holes or perforations.

In some embodiments, a patterned SLIPS layer is provided which provides for wicking or transport of a liquid phase through the layer through unfunctionalized portions of the layer. In some embodiments, a patterned SLIPS layer is capable of providing areas of patterning with smaller dimensions than is possible using pores, apertures, channels, wells, voids, holes or perforations in the substrate. In some embodiments, pores, apertures, channels, wells, voids, holes or perforations provide a faster conduit to remove liquid from the substrate than a patterned SLIPS layer.

In some embodiments, the patterned SLIPS layer is applied by removing selected areas of a SLIPS substrate. In further embodiments, the patterned SLIPS layer is formed by selectively applying SLIPS to an unfunctionalized surface.

In some embodiments, liquids (such as anti-inflammatory drugs, antibiotics, or other liquid treatments) can be injected through the non-functionalized surface and channels therein, without the need to remove the bandage. In other embodiments, liquids (such as anti-inflammatory drugs, antibiotics, or other liquid treatments) can be simply placed on the diffusive layer and wick through the non-functionalized surface and channels therein to reach the wound, without the need to remove the bandage.

In some embodiments, the absorbing material provides the ability to monitor a covered surface without disturbing the absorbing material. In certain embodiments, analytes of a liquid wicked from the surface through the non-functionalized surface are taken without the need to remove the absorbing material. In some embodiments, exudate, blood or plasma from a wound covered by an absorbing material is tested for infection. In other embodiments, the backing can be equipped with an indicator that changes color or undergoes another observable change to provide information about the state or condition of the wicked fluid. In other embodiments, for the patterned regions going through the article, one can replenish the lubricant itself, to produce a long-lasting articles (such as those applicable in burns, etc).

In certain embodiments, disclosed herein, the patterned material (whether a perforated SLIPS material, or a porous material having patterned hydrophilic holes, apertures, pores, channels, wells, voids or perforations) allows a SLIPS layer to act as a non-adhesive layer which can also allow flow of an aqueous phase, for drainage, absorption, sponge-like collection, perspiration or biosensing. Applications of such a design could be for biomedical wound dressing, tissue repair or bandage. Other applications include sponge-like materials, such as for diaper or surgical usage. A further application could be for protective layers against the skin, which allows the transport of perspiration through the non-adherent SLIPS layer.

In certain embodiments, the article further comprises a third region which comprises a liquid or solid adhesive. In some embodiments, said first and second regions are non-adhesive to at least one of skin, hair, dried blood or clotted blood. In some embodiments, the third region surrounds the first and second regions. In some embodiments, the article is a bandage our wound dressing, where the third region displays adhesive properties, and is configured to adhere to a patient's healthy tissues surrounding a wound, while the first and second regions are configured to contact the wound itself. In some embodiments, the first and second regions display absorbent wicking of exudate fluid from the wound (to an absorbent layer) while maintaining a non-adhesive surface which prevents adhesion of the wound tissue to the article. In some embodiments, the first and second regions comprise a SLIPS lubricant which is permeable to oxygen, to allow a high flux of oxygen to the wound tissue surface, to allow improved wound repair. In some embodiments, the article comprises a second side comprising an absorbent backing layer. In some embodiments, the absorbent backing layer is configured to wick and collect exudate fluid from a wound.

In some embodiments, at least one of the first and second sides comprises a protective sheet. In some embodiments, the protective sheet covers the absorbent backing layer. In some embodiments, the protective sheet is sacrificial and readily removable. In some embodiments, the protective sheet prevents at least one of the first, second or third regions from being exposed to external matter, such as liquids, dirt and debris. In some embodiments, the protective sheet forms a backing on at least one side of the article to seal the various components together. In some embodiments, the protective sheet prevents an adhesive region from adhering to objects prior to a user's removal of the protective sheet and use of the article. In some embodiments, the protective sheet is peeled off of the at least one side of the article to expose at least one of the first, second or third regions.

### Selective Deposition of Materials on Protected Surfaces

In one aspect of the present disclosure, a substrate is protected with a SLIPS surface, where selected regions are either unfunctionalized or define pores, apertures, channels, wells, voids, holes or perforations and materials are deposited in these selected regions (e.g., pixelation). Material (such as pigment or paint) can then be selectively deposited in these unfunctionalized regions, pores, apertures, channels, wells, voids, holes or perforations to create a predetermined pattern. Subsequently, a Liquid B can be applied to the substrate such that the unfunctionalized regions, pores, apertures, channels, wells, voids, holes or perforations are covered by Liquid B, and the entire surface is then protected by a substantially uniform SLIPS surfaces. Particular applications may include selective deposition of images on bank notes which are then protected from dust, oil, fingerprints, or other contaminants.

In certain embodiments, a surface is described herein is provided which possesses enhanced transparency at desired wavelengths. In certain embodiments, the roughened surface and Liquid B can be selected to have similar refractive indices so that the combination of roughened surface and Liquid B forms a transparent material in wavelengths, such as visible, infrared, or UV wavelengths. In this way, a protective SLIPS surface can be used as an anti-graffiti surface, being deposited on a building, statue, public infrastructure, sign or image (such as a road sign, banner, painting, photograph or billboard) to prevent the building, statue, public infrastructure, sign or image from collecting dust, oils, fingerprints, or other contaminants.

As used herein, “similar indices of refraction” means to have indices of refraction which can be differed from each other at least by ˜0.3. In certain embodiments, due to their substantially similar indices of refraction, SLIPS can be substantially transparent in desired ranges of wavelengths (e.g., UV, visible, infrared, and the like wavelengths), such as more than 70%, 80%, 90% or even 95% transparent.

In certain embodiments, SLIPS can be used for anti-graffiti purposes as they resist wetting of oil-based/water-based spray paints. Even when the paints solidify onto the SLIPS, the paints have very low adhesion to the surfaces which can be removed easily with adhesion tapes and the like. In addition, the solidified paints can also be removed by regular solvents, such as acetone without leaving traces of residues.

In certain embodiments, the surface of the construction materials can be roughened to provide a porous surface (i.e., roughened surface). Then, Liquid B that can repel contaminants, such as water-based spray paint, oil-based spray paint, rain, and the like can be selected. Then, the roughened surface can be infiltrated with Liquid B to form an ultra-smooth layer of Liquid B thereon. In certain embodiments, a reservoir that can replenish any loss of Liquid B can be provided.

Additional criteria that may be particularly important for applications in this category include shear-resistance, self-healing, and anti-wetting and anti-adhesive. Hence, Liquid B and the roughened surface can be selected to provide all or optimized combination of these characteristics.

In certain embodiments, the roughened surface can be selected from fluorosilanized materials and Liquid B can be selected from perfluoropolyether.

### Liquid Adhesives and Permeable/Non-Permeable Solid Backings

The low stick, low adhesion properties of SLIPS, while providing many advantages and uses, provides a challenge when it is desired to secure or adhere this otherwise low stick substrate to a base. A method and related article providing good adhesion of a SLIPS surface to its underlying base is described.

In another aspect of the present disclosure, a solid matrix comprising a SLIPS surface and a liquid adhesive backing is provided by selective displacement of lubricant. In certain embodiments, a porous solid having layers of different surface energy is provided, wherein one (upper) layer has matching surface energy to Liquid B (but does not have matching surface energy to Liquid A), while the other (lower) layer has matching surface energy to Liquid A (but does not having matching surface energy to Liquid A). Note that in this instance the heterogeneous surface energy properties are vertically and not laterally distributed. Accordingly, in certain embodiments, the material vertically heterogeneous SLIPS substrate can be treated with a non-adhesive Liquid B, while an adhesive Liquid A displaces the portion of Liquid B in contact with the layer of the material which has matching surface energy to Liquid A. This embodiment therefore provides a 2-layer material which can adhere to a secondary substrate on one side, while providing an outer SLIPS surface. In certain embodiments, Liquid A is chosen such that it can polymerize to form permanent bonding with the secondary solid substrate. In some embodiments, Liquid A is PDMS. In some embodiments, the substrate and liquids are optically matching such that the solid matrix is transparent. In some embodiments, the solid matrix is used as a sign cover to provide a SLIPS covering which does not compromise the optical clarity of the sign. In some embodiments, the 2-layer porous solid is provided by partial etching of one side of a SLIPS material such that one side is SLIPS and the other is unfunctionalized and can be treated with adhesive Liquid A.

FIG. 4 shows an exemplary embodiment of this aspect of the present disclosure, wherein a 2-layer porous solid that is composed of two different types of materials is first wetted by Liquid B. In the embodiment shown in FIG. 4, the substrate is made up of two different porous materials, Porous Material 1 and Porous Material 2. The surface properties may differ, for example, by providing different surface modification to either side of the substrate or by adhering two layers of different properties in facing relationship. The substrate is then infiltrated with Liquid B. Liquid B and Porous Material 1 have matching surface energy such that the liquid will be stably attached within the porous material, while Liquid B and Porous Material 2 do not have matching surface energy. By way of example, the substrate is a porous sheet that is treated to have a surface energy affinity to a lubricating liquid used to form the SLIPS surface. The article is then exposed to Liquid A that has a greater affinity for Porous Material 2. Liquid B that is trapped, but not stably attached, within the Porous Material 2 will be displaced. In certain embodiments, Liquid A can be a liquid epoxy precursor. Liquid A is then cured to form a solid backing. In some embodiments, Liquid B forms a meta-stable attachment to Porous Material 2. A meta-stable state is created when the lubricant's low surface tension wets the surface but a “lock in”, that is, the energetic minimum situation is not supported by the surface chemistry. As a result, the SLIPS state will eventually break down upon addition of a second liquid. However, this may take time, and the surface is in a SLIPS state until the stable surface liquid is disrupted. Thus, a meta-stable slips surface can be created even though the conditions for thermodynamic stability are not satisfied. A meta-stable state could also be created on a surface on which the supporting roughness is not high enough to allow a stabilized liquid layer (SLIPS) to form. In the embodiment shown in FIG. 4, Liquid B and Porous Material 1 have matching surface energy (i.e. ΔE₁>0 and ΔE₂>0) such that the liquid will be stably attached within the porous material, while Liquid B and Porous Material 2 do not have matching surface energy (i.e. ΔE₁<0 and/or ΔE₂<0), therefore with suitable Liquid A, Liquid B that is trapped within the Porous Material 2 will be displaced. Liquid A is then cured to form a solid backing.

### EXAMPLES

### Example 1

### Preparation of a Boehmite Surface From Aluminum

Aluminum metal is sandblasted at 30 psi using 120 grit sand. The resulting sandblasted metal is ultrasonicated in acetone for 15 minutes, dried, and boiled in distilled water for 10 minutes to produce a Boehmite surface.

### Example 2

### Preparation of a Sol-Gel Alumina-Based Boehmite Coating

A solution-based mixture can be used to fabricate SLIPS on arbitrary metal or non-metal substrates. The mixture can be applied by various application methods including spraying, dip coating, painting, spin coating, printing, drop casting, etc. Such mixtures can include sol-gel precursors to metal oxides, metal hydroxides, metal oxy hydroxides, or dispersions containing metal oxides, metal hydroxides, metal oxy hydroxides, where the metal component can include Mg, Al, Si, Ca, Ti, V, Cr, Mn, Fe, Co, Ni, Cu, Zn, Sr, Y, Zr, Nb, Mo, Ru, In, Sn, Ba, Hf, Ta, W, Os, Pb, Bi, La, Ce, Pr, Nd, Sm, Eu, Gd, Tb, Dy, Ho, Er, Tm, Yb, Lu, or the mixtures thereof. The sol-gel precursor can be deposited on arbitrary shapes, and then converted into a corresponding metal oxide, metal hydroxide, or metal oxy hydroxide, or salts. In some embodiments, A boehmite coating can be formed via a sol-gel process on non-aluminum substrates. Boehmite (a.k.a. Aluminum Oxide Hydroxide or AlO(OH)) is a crystalline form of aluminum oxide that can provide a high porosity, high surface roughness morphology. The boehmite coating can be formed on a wide range of substrates to provide uniform nanostructure as the roughened substrate for SLIPS. A transparent thin film of sol-gel derived alumina can be applied to various substrate surfaces—for example, glass, stainless steel, polymers (Polystyrene (PS). Poly(methyl methacrylate) (PMMA), polycarbonate, polysulfone, polyurethane, epoxy, polyolefins, Polyethylene terephthalate (PET), polyvinyl chloride (PVC), etc.)—using solution based deposition (spin coating, dip coating, spray coating) and vapor phase deposition (CVD, ALD, PVD) at temperature ranges from room temperature to 400®C.

In some embodiments, sol-gel alumina derived coatings on various materials can be patterned into nanostructured SLIPS and unstructured regions by using photo-curable sol-gel alumina precursors. The photo-patternable sol-gel boehmite method can generate topographical contrast by controlling the deposition of nanostructured materials. For example, the methacrylate group in 2-(methacryloyloxy) ethyl acetoacetate can be polymerized and crosslinked with an added crosslinker by photo-initiated radical generator, while the acetoacetate group can provide strong coordination to aluminum metal by replacing the alkoxy groups of the aluminum metal precursor:

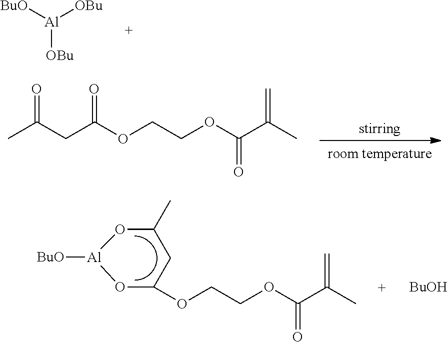

One exemplary sol-gel solution is a mixture of aluminum-tri-tert-butoxide+2-(methacryloyloxy) ethyl acetoacetate and a photoinitiator (e.g. DAROCUR 1173) ethylacetoacetate+2-propanol+water, to give photopolymerizable functions in order to created only certain regions of gel to be formed. An additional crosslinker, such as ethylene glycol dimethacrylate, may be added to improve mechanical properties of the coating layer.

When exposed to irradiation, the sol gel will photocure. If exposed to a pattern of light, the sol gel will cure in a pattern to provide a gel in only certain regions. This allows for microprinting and patterning of the boehmite-SLIPS surface to provide the heterogeneous surface for use in the applications exemplified herein. The pattern was generated using uv exposure to 100 W i-line for 120 s through a photomask and subsequent development in 2-propanol for 60 s. The photo-patterned sol-gel alumina was then converted to Boehmite by reacting with DIW at 100° C. for 10 min.

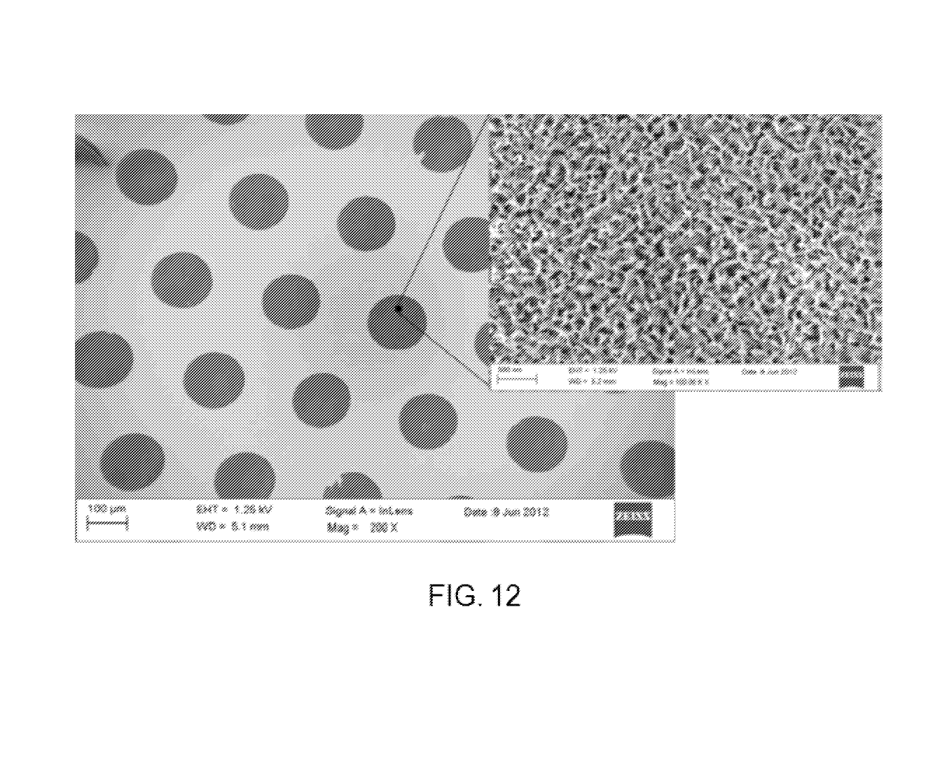

FIG. 12 contains SEM images of photo-patterned sol-gel alumina-derived SLIPS on a glass slide. The inset is a magnified view of the boehmite morphology. The dark circular areas correspond to SLIPS where the boehmite nanostructures are shown at higher magnification on the right. Scale bars are 500 μm and 500 nm, respectively. The patterned coating was prepared by spin coating a photo curable sol-gel alumina precursor solution layer on a glass substrate. After spin coating the photocurable sol-gel alumina precursor solution, the pattern was generated by UV exposure to 100 W i-line for 120 s through a photomask and subsequent development in 2-propanol for 60 s. The photo-patterned sol-gel alumina was then converted to Boehmite by reacting with deionized water at 100° C. for 10 minutes. Further detail on the use of metal containing substrates for the formation of SLIPS surfaces can be found in co-pending application entitled “SLIPS Surface Based on Metal Containing Compound,” filed on even date herewith and incorporated in its entirety by reference.

### Example 3

### Preparation of SLIPS with Patterned Wettability, Pure Smooth Surface

A smooth silicon/glass substrate is first chemically functionalized with heptadecafluoro-1,1,2,2-tetrahydrodecyltrichlorosilane (available from Gelest, Inc.) by vapor deposition for at least 24 hours. The chemically functionalized silicon/glass are then selectively patterned by shadow mask technique through the use of physical or chemical etching methods, such as oxygen plasma. The chemically functionalized area that is exposed to the oxygen plasma is selectively removed, rendering the glass material. After the selective patterning step, the silicon/glass are infused with a lubricating fluid, such as perfluorinated fluids (e.g., perfluoropolyether and the like). The regions with strong chemical affinity (the fluoro-silanized region) with the lubricant perform as SLIPS, and the regions with weak chemical affinity (i.e. glass region) with the lubricant are displaced by foreign, immiscible fluid, such as aqueous liquids or their complex mixtures (e.g., blood).

### Example 4

### Preparation of SLIPS with Patterned Wettability, Roughened Surface

A smooth silicon/glass substrate is deposited with inverse opal with long range ordered porous structures of silica, which is produced by evaporative co-assembly method of sacrificial polymeric colloidal particles together with a hydrolyzed silicate sol-gel precursor solution. This method generates a crack-free porous surface on the order of centimeters or larger, with pore sizes of about 100 nm to about 1000 nm and porosity of about 75%. (See Hatton, et al., Proc. Natl. Acad. Sci. 107, 10354-10359, 2010 and U.S. patent application Ser. No. 13/058,611, filed on Feb. 11, 2011, now US 2011/0312080, the contents of which is incorporated by reference herein in its entirety). With the porous inverse opal, the material is then chemically functionalized with heptadecafluoro-1,1,2,2-tetrahydrodecyltrichlorosilane (available from Gelest, Inc.) by vapor deposition for at least 24 hours. The chemically functionalized porous membrane is then selectively patterned by shadow mask technique through the use of physical or chemical etching methods, such as oxygen plasma. The chemically functionalized area that is exposed to the oxygen plasma is selectively removed, rendering the glass material. After the selective patterning step, the porous membranes are infused with a lubricating fluid, such as perfluorinated fluids (e.g., perfluoropolyether and the like). The regions with strong chemical affinity (the fluoro-silanized region) with the lubricant perform as SLIPS, and the regions with weak chemical affinity (i.e. glass region) with the lubricant are displaced by foreign, immiscible fluid, such as aqueous liquids or their complex mixtures (e.g., blood).

### Example 5

### Preparation of SLIPS with Patterned Wettability, Roughened Surface

A smooth silicon/glass substrate is roughened by photolithography process followed by chemical/physical etching to selectively remove the glass/silicon materials. The roughened material is then chemically functionalized with heptadecafluoro-1,1,2,2-tetrahydrodecyltrichlorosilane (available from Gelest, Inc.) by vapor deposition for at least 24 hours. The chemically functionalized structured substrates are then selectively patterned by shadow mask technique through the use of physical or chemical etching methods, such as oxygen plasma. The chemically functionalized area that is exposed to the oxygen plasma is selectively removed, rendering the glass material. After the selective patterning step, the substrates are infused with a lubricating fluid, such as perfluorinated fluids (e.g., perfluoropolyether and the like). The regions with strong chemical affinity (the fluoro-silanized region) with the lubricant perform as SLIPS, and the regions with weak chemical affinity (i.e. glass region) with the lubricant are displaced by foreign, immiscible fluid, such as aqueous liquids or their complex mixtures (e.g., blood).

### Example 6

### Preparation of SLIPS with Patterned Wettability, Porous Bulk Material

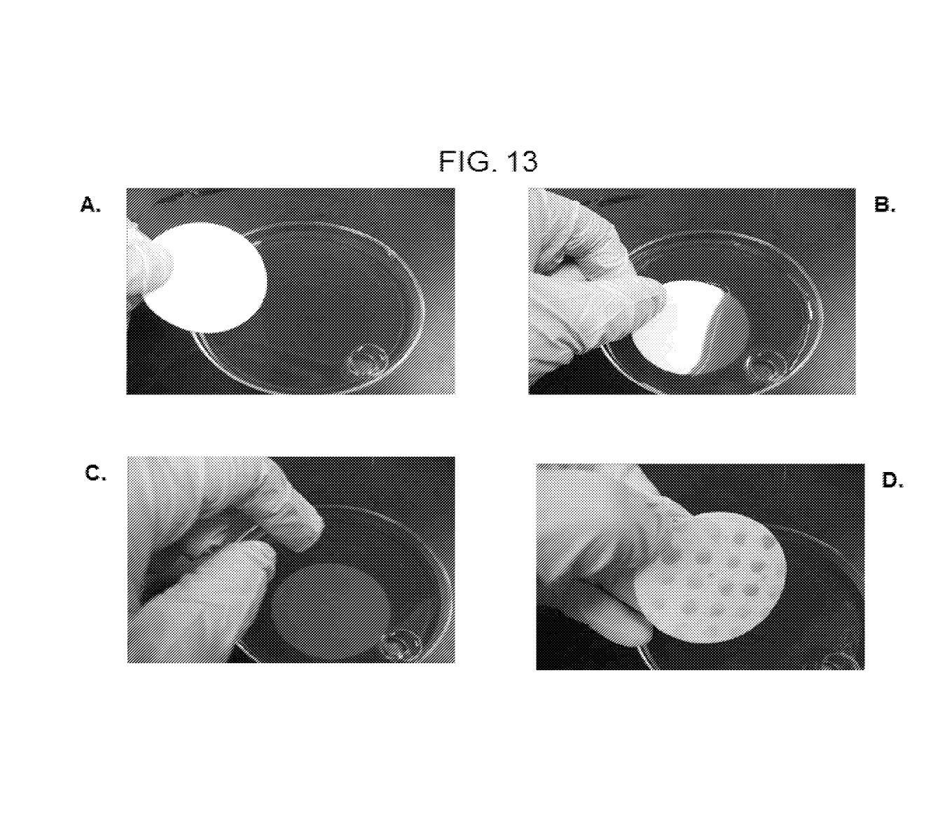

A porous fiber glass membrane (with pore size on the order of 200 nm or larger and membrane thickness of about 0.5 mm, available from Sterlitech Corporation, WA) is first chemically functionalized with heptadecafluoro-1,1,2,2-tetrahydrodecyltrichlorosilane (available from Gelest, Inc.) by vapor deposition for at least 24 hours. The chemically functionalized porous membrane is then selectively patterned by shadow mask technique through the use of physical or chemical etching methods, such as oxygen plasma. The chemically functionalized area that is exposed to the oxygen plasma is selectively removed, rendering the glass material. After the selective patterning step, the porous membrane is infused with a lubricating fluid, such as perfluorinated fluids (e.g., perfluoropolyether and the like). The regions with strong chemical affinity (the fluoro-silanized region) with the lubricant perform as SLIPS, and the regions with weak chemical affinity (i.e. glass region) with the lubricant are displaced by foreign, immiscible fluid, such as aqueous liquids or their complex mixtures (e.g., blood). The regions with weak chemical affinity with the lubricant are then selectively displaced by dyed water as shown in FIGS. 13A-D.

### Example 7

### Preparation of SLIPS with Patterned Wettability, Laminated Porous Materials

A porous Teflon membrane laminated with porous polypropylene membrane (with pore size on the order of 200 nm or larger and membrane thickness of about 0.5 mm, available from Sterlitech Corporation, WA) is infused with a lubricating fluid, such as perfluorinated fluids (e.g., perfluoropolyether and the like). The regions with strong chemical affinity (the fluoro-silanized region, i.e. Teflon) with the lubricant perform as SLIPS, and the regions with weak chemical affinity (i.e. polypropylene region) with the lubricant are displaced by foreign, immiscible fluid, such as polydimethylsiloxane (PDMS). The displaced layer is used as a liquid adhesive or solid adhesive (for cured PDMS) to stick the functional SLIPS layer onto other surfaces.

### Example 8

### Preparation of SLIPS-Based Non-Adhesive Bandage

A non-adhesive bandage or wound dressing which comprises a SLIPS layer with patterned wettability and is capable of absorbent wicking and significant oxygen transport to the tissue surface is provided. A porous PTFE layer having an array of 1.0 mm diameter channels disposed through the thickness of the substrate is infiltrated and saturated with perfluorocarbon liquid (Fluorinert FC70, 3M). An absorbent backing layer of hydrophilic tissue (Kimwipe) is placed behind the SLIPS layer. This combination comprises a SLIPS bandage, suitable for placing in contact with an exposed wound surface.

### Example 9

### Preparation of Transparent SLIPS Coating on Glass With Colloidal Monolayers

### Monolayer Formation on Glass.

Colloidal monolayers are crystallized on the air water interface of a Langmuir trough following a protocol from literature (Vogel et al. Adv. Funct. Mater. 2011, 21, 3064). In brief, a colloidal dispersion in 1:1 water/ethanol (solid content approx. 2.5%) is spread onto the interface via a glass slide until a third of the trough's surface is covered. The available surface area is subsequently reduced by barriers until the complete surface is covered with the colloidal monolayer. Transfer to glass substrate (deposited into the water subphase prior to the colloids' addition) is achieved by lowering the surface level until the monolayer is gently placed onto the substrate. After drying, a closed-packed monolayer uniformly covers the substrate. Alternatively, the same process can be used without a Langmuir trough by floating the colloidal dispersion onto a crystallization dish until the surface is completely covered with a colloidal monolayer (Vogel et al. Macromol. Chem. Phys. 2011, 212, 1719). There are many existing methods to crystallize monolayers on solid substrates (for a review, please refer to: Vogel et al. Soft Matter 2012, 8, 4044) that could be used as well.

### Formation of the Inverse Monolayer

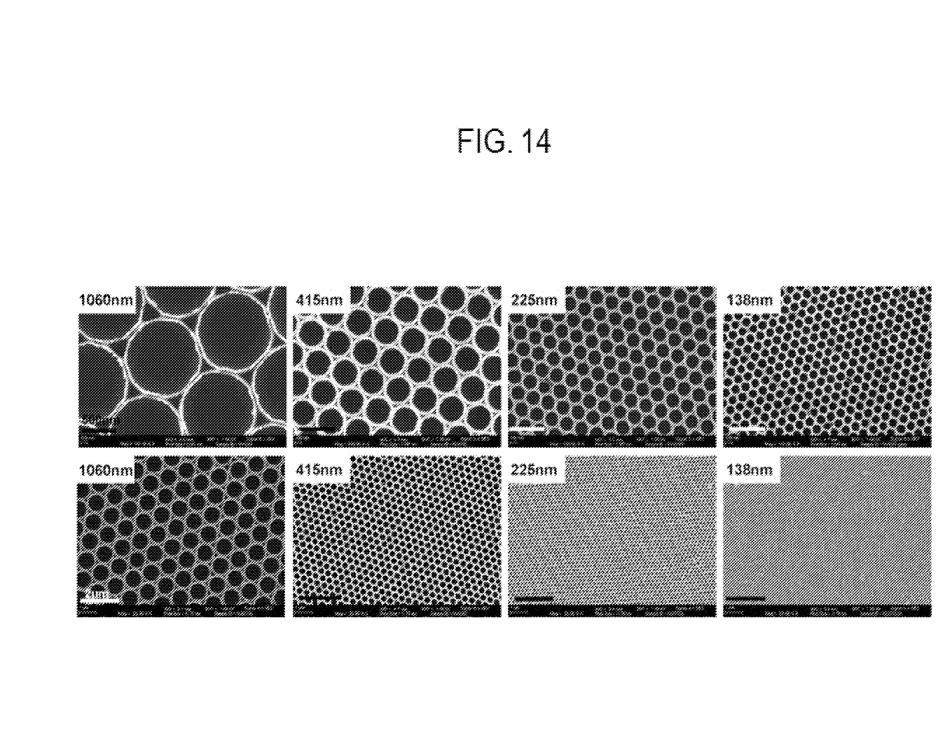

A solution of Tetraethylorthosilicate (TEOS), HCl (0.1 mol/L) and ethanol with weight ratios of 1:1:1.5 is prepared and stirred for 1 h. Then, it is diluted with ethanol (dilution ratios see Table 1) and spin-coated onto the monolayer-covered substrate (3000 rpm, 30 s, acc. 500). The colloids are removed by combustion at 500° C. (ramped from RT to 500° C. for 5 h, 2 h at 500° C.). The process would also work with any other inorganic sol-gel processes as well as with different polymers or nanoparticles that could be used to backfill the monolayer. Besides spin-coating, drop casting, dip coating, spray coating or chemical vapor deposition could also be used. The colloids can also be removed at room temperature using organic solvents (tetrahydrofuran, toluene, etc.). Examples of these surface structures are shown in FIG. 14.

TABLE 1
Mixture ratios between TEOS and Ethanol used for spincoating

| Colloid | TEOS:Ethanol ratio |
|---|---|
| size | (volume) |
| 1000 nm | 1:0.9 |
| 415 nm | 1:4 |
| 225 nm | 1:6 |
| 138 nm | 1:9 |

### Fluorosilanization

Fluorosilanization is carried out by vapor-phase deposition of (1H,1H,2H,2H-tridecafluorooctyl)-trichlorosilane for 24 h at reduced pressure and room temperature. Prior to silanization, the substrates are cleaned in acid piranha (3:1 sulfuric acid:hydrogen peroxide; WARNING: reacts very violently with organic materials) and plasma treated with oxygen plasma for 10 min. The same results can also be achieved with different fluorosilanes. Required times can be reduced by changing the temperature. Acid and plasma cleaning are provide redundant cleaning and could be simplified or omitted.

### Lubrication

10 μl/cm2 Krytox 100 is added to the substrate and uniform coverage is achieved by tilting. Excess lubricant is removed by vertical placement of the substrates. Further detail on the preparation of SLIPS surfaces using colloidal monolayers is found in co-pending International Patent Application entitled SLIPPERY LIQUID-INFUSED POROUS SURFACES HAVING IMPROVED STABILITY filed on even date herewith, the contents of which are incorporated by reference.

### Example 10

### Preparation of Patterned SLIPS Substrates Using Colloidal Deposition

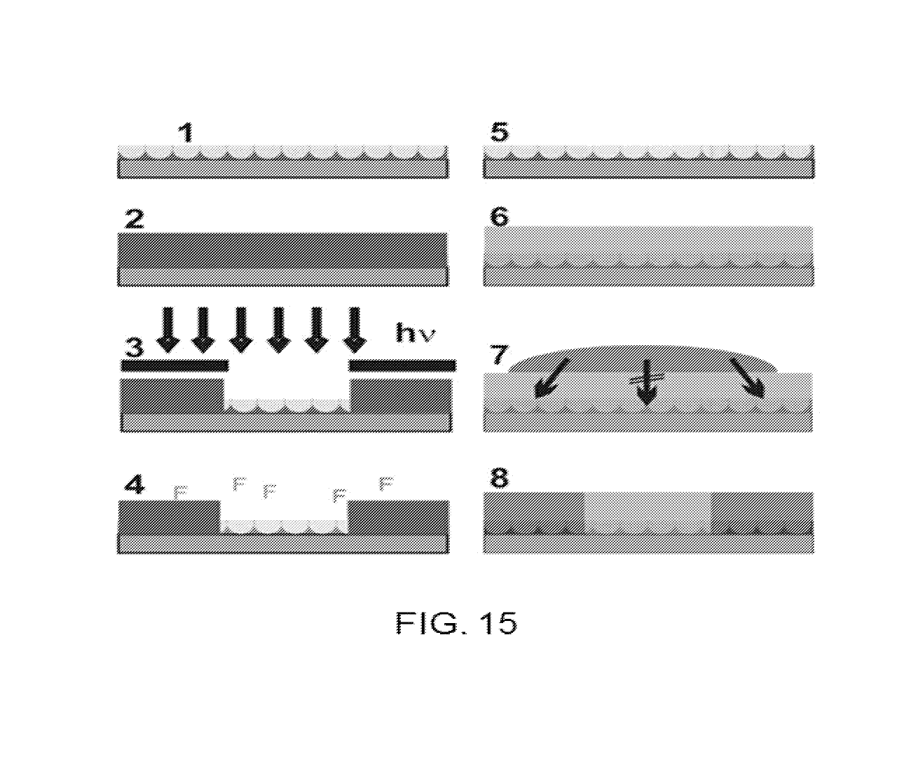

The porous substrates prepared from colloidal templating can be applied to conventional photolithographic processes prior to silanization. A conventional photoresist (S1818, positive tone resist) is spincoated (4000 rm, 60 s) on the unsilanized inverse monolayer substrate. Illumination with UV light following the recipe of the photoresist manufacturer (Shipley Company, Marlborough, Mass.) with a irradiation dose of 200 mJ/cm² followed by development of the structures (Developer MF319, 60 s). The substrate, consisting of areas covered by photoresist and free areas is plasma treated for 5 min and then fluorosilanized similar to non-patterned substrates. After silanization, the resist is washed off the substrate (Remover PG 1165). Application of lubricant then produces patterned SLIPS surfaces as the conditions for stable SLIPS state is only fulfilled in the fluorosilane-functionalized surface regions. Such structures can be patterned for preparation of a patterned SLIPS surface. For example, as shown in FIG. 15, an inverse monolayer substrate (1) can be coated with photoresist (2), irradiated and developed to create the desired surface pattern (3). Vapor-phase silanization can be applied to create fluorinated surface functionalities at the exposed surface regions (4); the protected surface regions can remain unfunctionalized after removal of the photoresist (5). Due to its low surface energy, addition of lubricating liquid can first create a homogeneous liquid film on the surface (6). Then, addition of a second repellent liquid (7) can lead to a selective replacement of the lubricating liquid at the unfunctionalized surface areas (8).

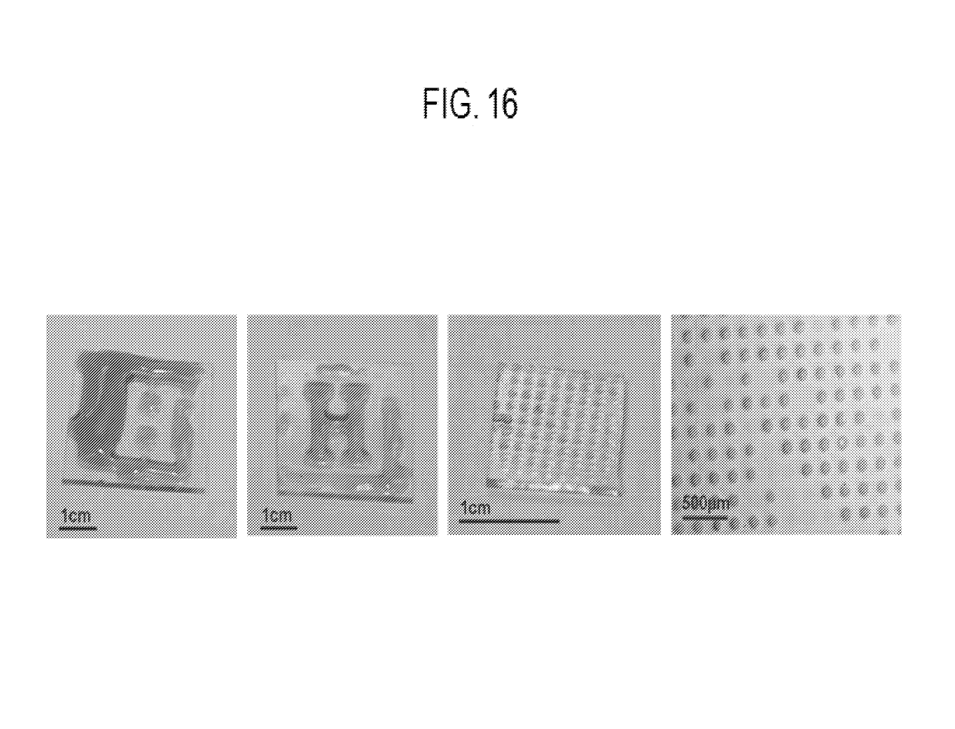

The uniformity and low height of the surface structures enables the application of conventional photolithographic processes to prepare locally confined patterned SLIPS regions (see FIG. 15). The process is based on the creation of surface functionality patterns. The lubricating liquid is only locked-in firmly in areas with matching surface chemistry, while it will be replaced by a second liquid in areas with different surface chemistry. However, due to its low surface energy, addition of the lubricating liquid leads to a homogeneous lubricating liquid film covering the entire coating. Upon the addition of a second liquid, a metastable state can be created in which the lubricating liquid layer is not immediately disrupted by the liquid (see state 6 in FIG. 15). Eventually, the energetically most favorable state is reached as the liquid displaces the lubricating liquid layer in non-functionalized surface regions. Stable SLIPS surface regions defined by the fluorinated surface chemistry remain wetted by the lubricating liquid only without being displaced by the applied test liquid to be repelled. The superior performance of the SLIPS-based approach to create patterns of liquids on solid substrates is shown in FIG. 16. As shown, several test liquids are successfully confined to designated surface regions based on matching surface chemistry.

Those skilled in the art would readily appreciate that all parameters and configurations described herein are meant to be exemplary and that actual parameters and configurations will depend upon the specific application for which the systems and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. The present invention is directed to each individual feature, system, or method described herein. In addition, any combination of two or more such features, systems or methods, if such features, systems or methods are not mutually inconsistent, is included within the scope of the present invention.

What is claimed is:

1. A liquid-coated substrate, comprising:
a substrate having a spatially heterogeneous surface pattern, said spatially heterogeneous pattern comprising:
at least one first region having surface characteristics that provide a stable liquid film with a first lubricating liquid; and
at least one second region having one of surface characteristics that provide an unstable liquid film with said first lubricating liquid or apertures or holes that provide liquid conduits through the substrate; and
a first lubricating liquid disposed over at least a portion of said first region to provide an immobilized liquid layer.

2. The liquid-coated substrate of claim 1, wherein the liquid-coated substrate is a non-adherent article capable of absorbent wicking, wherein:
the spatially heterogeneous surface pattern is disposed through at least a portion of the thickness of the substrate;
wherein the first lubricating liquid is substantially immobilized in and on the at least one first region of the substrate to form a stabilized liquid overlayer, said overlayer providing a non-adherent surface;
wherein the first lubricating liquid has a lower affinity for the second region of the substrate as compared to the at least one first region or the second region comprises a hole or aperture that does not retain the first lubricating liquid; and
wherein the second region is capable of wicking a wicking liquid into or through said second region.

3. The liquid-coated substrate of any preceding claim, wherein said second region comprises apertures or holes.

4. The liquid-coated substrate of claim 3, wherein the substrate comprises a perforated material with large holes that provide liquid conduits through the material.

5. The liquid-coated substrate of any preceding claim, wherein the substrate is selected from a flat solid, a textured solid, a porous solid and combinations thereof.

6. The liquid-coated substrate of any preceding claim, wherein at least one of the first region or second region is chemically functionalized to provide surface characteristics that provide either a stable liquid film or an unstable liquid film with the first lubricating liquid.

7. The liquid-coated substrate of any preceding claim, further comprising an absorbent backing.

8. The liquid-coated substrate of any one of claims 2-7, wherein the wicking liquid is immiscible in the first lubricating liquid.

9. The liquid-coated substrate of any one of claims 2-8, wherein the wicking liquid has a greater affinity for the apertures than the first lubricating liquid.

10. The liquid-coated substrate of any one of claims 2-9, wherein the wicking liquid comprises at least one of proteins, bacteria, cells or non-biological entities.

11. The liquid-coated substrate of claim 2-10, wherein the first region is hydrophobic, and the second region comprises chemically etched hydrophilic regions or holes allows the wicking and transport of an aqueous phase.

12. The liquid-coated substrate of any one of claims 2-10, wherein the substrate is porous and wherein the second region is capable of wicking a wicking liquid into or through said porous substrate.

13. The liquid-coated substrate of claim 2-12, wherein the article is configured and arranged to collect the absorbed/wicked liquid without the removal of the article.

14. The liquid-coated substrate of claim 2-12, wherein the article is configured and arranged to inject/wick through the conduits certain liquids to reach the target area without the removal of the article.

15. The liquid-coated substrate of any one of claims 3-12, wherein the apertures have an average diameter or width of at least about 200 nm, or at least about 0.2 mm, or at least about 0.25 mm, at least about 0.5 mm, at least about 0.75 mm, at least about 1.00 mm, at least about 1.25 mm, at least about 1.50 mm, at least about 1.75 mm, or at least about 2.00 mm.

16. The liquid-coated substrate of any preceding claim, wherein the liquid-coated substrate is incorporated into a wound dressing, diaper, surgical dressing, sanitary pad or antiperspirant dressing.

17. The liquid-coated substrate of any preceding claim, wherein the article comprises a liquid-coated substrate surrounded by an adhesive area.

18. The liquid-coated substrate of claim 17, wherein spatially heterogeneous surface is non-adhesive to at least one of skin, hair, dried blood or clotted blood.

19. The liquid-coated substrate of any preceding claim, wherein the liquid-coated substrate is anti-bacterial.

20. The liquid-coated substrate of any preceding claim, wherein the liquid-coated substrate is pathogen-resistant.

21. The liquid-coated substrate of any preceding claim, wherein the liquid-coated substrate is anti-adhesion against biological cells.

22. The liquid-coated substrate of any preceding claim, wherein the liquid-coated substrate has anti-coagulating properties.

23. The liquid-coated substrate of any preceding claim, wherein the substrate is a flat solid, the at least one first region comprising chemical functionalization.

24. The liquid-coated substrate of any one of claims 1-22, wherein the substrate is a roughened solid, the at least one first region comprising at least one of chemical functionalization or the roughened solid.

25. The liquid-coated substrate of any one of claims 1-22, wherein the substrate is a porous solid, the at least one first region comprising at least one of chemical functionalization or the porous solid.

26. A liquid-coated substrate, wherein the liquid-coated substrate is a non-adhesive article capable of absorbent wicking, the liquid-coated substrate comprising:
a substrate;
at least one first region disposed on an upper portion of the substrate, said at least one first region having surface characteristics that provide a stable liquid film with a first lubricating liquid;
at least one second region disposed on the lower portion of the substrate, said at least one second region having surface characteristics that provide an unstable liquid film with said first lubricating liquid, wherein the at least one second region is capable of wicking a wicking liquid into or through said second region;
a first lubricating liquid disposed over at least a portion of said at least one first region, wherein:
the first lubricating liquid is substantially immobilized in and on the at least one first region of the substrate to form a stabilized liquid overlayer, said overlayer providing a non-adherent surface;
the first lubricating liquid has a lower affinity for the at least one second region as compared to the at least one first region; and
said wicking liquid is immiscible in the first lubricating liquid and has a lower affinity for the at least one first region as compared to the at least one second region.

27. The liquid-coated substrate of claim 26, wherein the upper porous layer of the substrate is hydrophobic, oleophobic or omniphobic and the lower porous layer of the substrate is hydrophilic or oleophilic or omniphilic.

28. The liquid-coated substrate of any one of claims 26-27, wherein the second liquid comprises a curable polymer.

29. The liquid-coated substrate of any one of claims 26-28, wherein the second liquid comprises an adhesive.

30. The liquid-coated substrate of any one of claims 26-29, further comprising a composite backing layer comprising a solid polymer infused in the lower porous layer of the substrate.

31. The liquid-coated substrate of any of the preceding claims, wherein the first lubricating liquid is optical refractive index-matched with the substrate.

32. The liquid-coated substrate of any of the preceding claims, wherein at least one of the first lubricating liquid or the second liquid is transparent to at least one of infrared, visible, or ultra-violet lights and wherein at least one of the first lubricating liquid or the second liquid is opaque to at least one of infrared, visible, or ultra-violet lights.

33. The liquid-coated substrate of any one of claims 31-32, wherein the liquid-coated substrate is suitable as an optical filter.

34. The liquid-coated substrate of any preceding claim, wherein the spatially heterogeneous surface pattern comprises at least one of a predetermined pattern, a symbol or a drawing.

35. The liquid-coated substrate of any one of claims 26-34, wherein said upper layer comprises a spatially heterogeneous surface pattern, said spatially heterogeneous pattern comprising at least one first region having surface characteristics that provide a stable liquid film with the first lubricating liquid.

36. The liquid-coated substrate of claim 35, said spatially heterogeneous pattern of said upper layer further comprising at least one second region having surface characteristics that provide an unstable liquid film with the first lubricating liquid.

37. The liquid-coated substrate of any one of claims 26-36, at least a portion of said lower layer having affinity for an adhesive.

38. The liquid-coated substrate of any one of claims 26-37, wherein at least one of the first side or the second side comprises an adhesive.

39. A liquid-coated substrate configured as a bandage to be applied to a wound, wherein the liquid-coated substrate is a non-adhesive article capable of absorbent wicking, the liquid-coated substrate comprising:
a substrate having a spatially heterogeneous surface pattern disposed through at least a portion of the thickness of the substrate, said spatially heterogeneous pattern comprising:
at least one first region having surface characteristics that provide a stable liquid film with a first lubricating liquid;
at least one second region having surface characteristics that provide an unstable liquid film with said first lubricating liquid; and
a first lubricating liquid disposed over at least a portion of said first region to provide an immobilized liquid layer;
an absorbent backing; and
a protective overlayer to protect the wound from the environment and to adhere to healthy tissue surrounding the wound;
wherein the first lubricating liquid is substantially immobilized in and on the at least one first region of the substrate to form a stabilized liquid overlayer, said overlayer providing a non-adherent surface;
wherein the first lubricating liquid has a lower affinity for the second region of the porous substrate as compared to the at least one first region; and
wherein the second region is capable of wicking a wicking liquid into or through said second region.

40. A liquid-coated substrate configured as fluid-transport devices, wherein the liquid-coated substrate is a non-adhesive article capable of fluid transport for biomedical and diagnostic purposes, the liquid-coated substrate comprising:
a substrate having a spatially heterogeneous surface pattern disposed through at least a portion of the thickness of the substrate, said spatially heterogeneous pattern comprising:
at least one first region having surface characteristics that provide a stable liquid film with a first lubricating liquid;
at least one second region having surface characteristics that provide an unstable liquid film with said first lubricating liquid; and
a first lubricating liquid disposed over at least a portion of said first region to provide an immobilized liquid layer;
wherein the first lubricating liquid is substantially immobilized in and on the at least one first region of the substrate to form a stabilized liquid overlayer, said overlayer providing a non-adherent surface:
wherein the first lubricating liquid has a lower affinity for the second region of the porous/roughened substrate as compared to the at least one first region; and
wherein the second region is capable of transporting fluids into or through said second region.

41. A liquid-coated substrate configured as selective patterning devices for biological species, such as proteins, DNA, RNA, viruses, cells, tissues, etc., wherein the liquid-coated substrate is a non-adhesive article capable of preventing adhesion from biological species, the liquid-coated substrate comprising:
a substrate having a spatially heterogeneous surface pattern disposed through at least a portion of the thickness of the substrate, said spatially heterogeneous pattern comprising:
at least one first region having surface characteristics that provide a stable liquid film with a first lubricating liquid;
at least one second region having surface characteristics that provide an unstable liquid film with said first lubricating liquid; and
a first lubricating liquid disposed over at least a portion of said first region to provide an immobilized liquid layer;
wherein the first lubricating liquid is substantially immobilized in and on the at least one first region of the substrate to form a stabilized liquid overlayer, said overlayer providing a non-adherent surface;
wherein the first lubricating liquid has a lower affinity for the second region of the porous/roughened substrate as compared to the at least one first region; and
wherein the second region is capable of selectively patterning biological species into or through said second region.

42. A method for absorbing a fluid, comprising:
providing the non-adhesive liquid-coated substrate of any of claims 1-38; and
exposing the liquid-coated substrate to a third liquid to be absorbed that is immiscible with the first lubricating liquid, wherein the third liquid fills the one or more apertures or second region of the liquid-coated substrate without displacing the stabilized liquid overlayer of the liquid-coated substrate to thereby retain the low adhesion properties of the liquid-coated substrate during absorption.

43. The method of claim 42, wherein the liquid-coated substrate is exposed to a wound.

44. The method of claim 43, further comprising disposing an oxygen-permeable lubricant between said liquid-coated substrate and said wound.

45. The method of claim 44, wherein said lubricant is selected from perfluorocarbons and silicone oils.

46. A method of adhering a liquid-coated substrate to an exposed surface of an object, the method comprising:
(a) providing a liquid-coated substrate, the liquid-coated substrate comprising a first region; the first region having surface characteristics that provide a stable liquid film with a first lubricating liquid; a first lubricating liquid disposed over at least a portion of said first region; and a second region surround the first region, the second region having surface characteristics that provide an adhesive; and
(b) contacting the portion of the liquid-coated substrate containing the first region to an exposed surface of the object, said contacting causing the second region of the liquid-coated substrate to adhere to the exposed surface of the object.
